(19) United States
(12) Patent Application Publication
(10) Pub. No.: US 2005/0004133 A1
(43) Pub. Date: Jan. 6, 2005

(54) Modulators of VR1 receptor

(76) Inventors: Lewis R. Makings, Encinitas, CA (US); Peter D.J. Grootenhuis, San Diego, CA (US); Dennis James Hurley, San Marcos, CA (US); Roger D. Tung, San Diego, CA (US); Andreas P. Termin, Encinitas, CA (US)

Correspondence Address: VERTEX PHARMACEUTICALS INC., 130 WAVERLY STREET, CAMBRIDGE, MA, 02139-4242, US

(21) Appl. No.: 10/861,788

(22) Filed: Jun. 4, 2004

Related U.S. Application Data
(60) Provisional application No. 60/476,457, filed on Jun. 5, 2003.

(30) Foreign Application Priority Data
Jun. 4, 2004 (WO) PCT/US04/17779

(51) Int. Cl. C07D 491/02; C07D 498/02; A61K 31/506; A61K 31/501; A61K 31/497

(52) U.S. Cl. 514/252.04; 514/255.05; 514/256; 514/301; 514/302; 544/238; 544/333; 544/405; 546/114; 546/115

(57) ABSTRACT

The present invention relates to compounds useful as modulators of the vanilloid receptor, and also provides pharmaceutically acceptable compositions comprising the compounds of the invention and methods of using the compositions in the treatment of various disorders.

### CROSS-REFERENCE TO RELATED APPLICATIONS

The present application claims priority under 35 U.S.C. § 119 to U.S. provisional application Ser. No. 60/476,457, filed Jun. 5, 2004, the entire disclosure whereof is incorporated herein by reference.

### TECHNICAL FIELD OF THE INVENTION

The present invention relates to compounds useful as modulators of the vanilloid receptor, and also provides pharmaceutically acceptable compositions comprising the compounds of the invention and methods of using the compositions in the treatment of various disorders.

### BACKGROUND OF THE INVENTION

The vanilloid receptor 1 (hereinafter “VR41”) is localized on sensory neurons and has been associated with disease related pain, such as, inflammatory pain, neuropathic pain, acute pain, chronic pain, post-operative pain, migraine, arthralgia, nerve injury, neurodegeneration, neuropathies, diabetic neuropathy, hyperactive urinary bladder, hypersensitive urinary bladder, urinary incontinence, interstitial cystitis, painful bladder disorders, irritable bowel syndrome, inflammatory bowel disease, inflammatory disease, asthma, chronic obstructive pulmonary disease, digestive tract ulcer, skin irritation, eye irritation, mucous membrane irritation. Pharmacological modulation of VR1 can result in prevention or treatment of these diseases.

The VR1 protein is a ligand-gated ion channel that can be activated by a broad range of stimuli. Stimuli (or agonists) for VR1 include the chili-pepper extract capsaicin, heat (>42° C.), protons, and a variety of endogenous lipids including but not limited to NADA, anandamide, and the eicosanoid 15-(S)-HETE (Gunthorpe, et al, TIPS 2002). Other agonists include the ultrapotent VR1 agonist resiniferatoxin (RTX). The endogenous or ‘natural’ activators of VR1 are thought to be heat, protons, and lipids like NADA, and current research suggests that VR1 may integrate multiple stimuli during inflammation to result in VR1 channel activation.

The VR1 channel is a member of a family of membrane-bound proteins known as TRP channels, and within the TRP family nomenclature, VR1 is known as TRPV1 (Gunthorpe et al, TIPS 2002). VR1 itself is also known as the “vanilloid receptor” and the “capsaicin receptor”. TRP channels have a 6-transmembrane domain topology. VR1 is most closely related by sequence homology to OSM-9 (thermal and osmotic sensor in C. elegans). Related TRP channels are generally less well characterized. These include TRPV2 (aka VRL1, activated by heat (>52° C.) but not capsaicin), TRPV4 (aka VRL2, localized in kidney and associated with osmotic control), TRPV5 and TRPV6 (intracellular calcium regulation), and the recently described “cold-receptor” CMR1 (McKemy, Neuhausser, Julius, Nature 2002).

Agonist-mediated activation of VR1 results in channel opening, and subsequent influx of calcium and sodium ions (PCa>PNa) into the sensory neurons expressing VR1. Influx of calcium and sodium ions serves a signaling role in the activation of these neurons by VR1 agonists.

The VR1 channel is expressed predominantly in small sensory neurons (e.g. DRG, cranial ganglia), most particularly in the small myelinated C-fibers that are thought to process or transmit painful sensory stimuli. VR1 is also localized and expressed in small sensory neurons that serve a sensory role in visceral tissues such as bladder. Localization of VR1 to sensory neurons that are ‘hard wired’ to pain pathways sensory supports a close association between VR1 activation and sensation of pain.

In addition to anatomical connections between VR1 and pain pathways, a strong correlation between activation of VR1 and the sensation of pain has been noted in humans and in animal studies. The simplest example of this is the burning sensation caused by exposure of human mucosa to chili peppers, or to purified extracts of peppers, namely the selective VR1 agonist capsaicin. Many other such examples exist including the pain associated with heating of the skin, and with tissue acidification, both stimuli strongly correlated with VR1 activation.

A VR1 knock-out mouse has been generated (D. Julius at UCSF), and has been characterized with a phenotype that is consistent with a role of VR1 in pain transmission.

Pharmacological inhibitors of VR1 activity have been reported, including the antagonists capsazepine, ruthenium red, and a broad assortment of other compounds functioning as classical VR1 receptor antagonists (Wang et al, Mol Pharmacol, 2002). These compounds have been shown to induce inhibition of VR1 activity using in vitro methods, such as electrophysiology, radioligand binding, and a variety of modern biochemical assays including fluorescence, etc.

In addition to direct blockade of VR1 activation via the use of classical receptor antagonists, functional blockade of VR1 channels has also been demonstrated by exploitation of VR1 agonist-induced receptor desensitization. During continuous or repeated exposures to agonists, the VR1 channel demonstrates strong desensitization, such that agonists induce functional inhibition of channel activation. For example, in vitro techniques have demonstrated that brief exposure of VR1 channels to the VR1 agonist capsaicin results in a rapidly-activating peak current, which is followed by dramatic reduction of the current despite the continued presence of capsaicin. Subsequent exposures to the agonist result in barely detectable currents, supporting the theory that activation of VR1 results in subsequent counter-intuitive blockade of channel activation.

This phenomenon has been leveraged therapeutically in the form of VR1 agonist(s) that are utilized in humans for pain relief. One example of this is the topical use of capsaicin creams, which induce an initial painful burning sensation (due to channel activation) and subsequent long-lasting relief of pain (due to desensitization). In addition, the VR1 agonists capsaicin and RTX have demonstrated utility in reducing bladder hyper-reflexia and bladder pain in humans after intra-bladder administration. The mechanism for bladder relief is thought to be induced by agonist-induced desensitization, and this effect appears to be long lasting, but associated with an initial pain response due to the “pungent” effects of VR1 agonism.

In vivo, a number of lines of evidence support the strategy that functional inhibition of VR1 activity holds therapeutic promise for reduction of pain. Examples of VR1 blockade resulting in reduction of pain come from both pure receptor antagonists, and from agonist-induced desensitization, both in humans and in animal studies. For example VR1 antagonists have been shown to be active as blockers of pain as measured in animal pain models Walker et al, JPET 2003). Many of these inhibitors are being developed for treatment of a variety of pain conditions, utilizing the underlying strategy that blockade of VR1 activity results in pain relief.

In addition, inhibition of pain has been demonstrated extensively in humans, using the agonist approach. For example, capsaicin creams are used extensively in human pain conditions such as trigeminal neuralgia, shingles, and inflammatory pain conditions. In addition, recent evidence from human clinical trials support the use of VR1 agonists in urinary incontinence and interstitial cystitis, via a catheter-based intravesicular bladder administration.

These data support the concept that functional blockade of VR1 channels will result in relief of pain in a variety of sensory neuron mediated pain syndromes, and in other syndromes in which sensory neurons are over-activated.

### SUMMARY OF THE INVENTION

It has now been found that compounds of this invention, and pharmaceutically acceptable compositions thereof, are useful as modulators of the ligand-gated ion channel VR1. These compounds have he general Formula I:

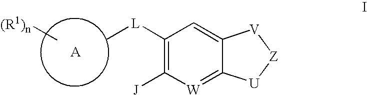

or a pharmaceutically acceptable derivative thereof, wherein Ring A, R¹, n, L, J, W, U, V, and Z are defined below.

This invention relates to VR1 receptor modulators, particularly VR1 receptor functional inhibitors, and to methods for using such modulators for the treatment of diseases including but not limited to pain, inflammatory pain, neuropathic pain, acute pain, chronic pain, post-operative pain, migraine, arthralgia, nerve injury, neurodegeneration, neuropathies, diabetic neuropathy, hyperactive urinary bladder, hypersensitive urinary bladder, urinary incontinence, interstitial cystitis, painful bladder disorders, irritable bowel syndrome, inflammatory bowel disease, inflammatory disease, asthma, chronic obstructive pulmonary disease, digestive tract ulcer, skin irritation, eye irritation, mucous membrane irritation.

### DETAILED DESCRIPTION OF THE INVENTION

1. General Description of Compounds of the Inventions

The present invention related to compounds of Formula I useful as VR1 receptor modulators:

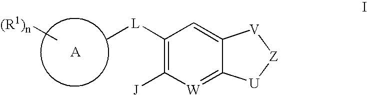

or a pharmaceutically acceptable salt thereof, wherein:

Z is C═O, or N;

V and U are independently selected from the group consisting of O, S, C═O, —CH₂—, —NR²—, wherein R² is —H, C₁₋₄alkyl, benzyl, or

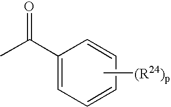

wherein R^{2A} is C₁₋₆alkyl, and p is 0-5;

W is C or N;

J is hydrogen, halo, or C₁₋₄alkoxy;

L is —NH—C(O)—(CH₂)_{q}—, —C(O)—NH—(CH₂)_{q}—, —NH—(CH₂)_{q}—, —(CH₂)_{q}NH— (where q is 0 to 2), —S(O)₂NH—, —NH—C(O)—NH—, or —CHR³—C(O)—NH—, wherein R³ is C₁₋₆alkyl; Ring A is C₃₋₇cycloalkyl, phenyl, pyrrolyl, pyrazolyl, imidazolyl, furanyl, thienyl, oxazolyl, isoxazolyl, triazolyl, isothiazolyl, pyridinyl, pyrimidinyl, pyridazinyl, pyrazinyl, piperidinyl, indolyl, indazolyl, benzotriazolyl, benzopyrazolyl, benzimidazolyl, benzthiazolyl, benzisothiazolyl, benzoxazolyl, benzisoxazolyl, benzotriazolyl, thiadiazolyl, benzothienyl, or triazinyl;

R¹ is independently selected from the group consisting of C₁₋₆alkyl, C₁₋₆alkoxy, -halo, —CF₃, —O—CF₃, —NH₂, —NH(C₁₋₄alkyl), —N(C₁₋₄alkyl)₂, C₁₋₄thioalkyl, —C(O)H, —C(O)OH, —C(O)—R^{1A}, C(O)OR A, wherein R^{1A} is C₁₋₆alkyl, and

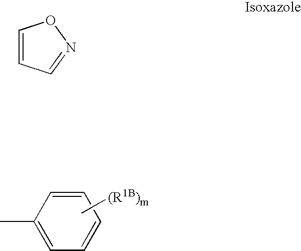

wherein R^{1B} is independently selected from C₁₋₆alkyl, C₁₋₆alkoxy, cyano, and -halo; and m is 0-5; and

n is 0-5.

2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The terms “aliphatic”, “aliphatic group” or “alkyl” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms, for example C₁₋₂₀alkyl. In some embodiments, aliphatic groups contain 1-10 aliphatic carbon atoms, for example C₁₋₁₀alkyl. In other embodiments, aliphatic groups contain 1-8 aliphatic carbon atoms, for example C₁₋₈alkyl. In still other embodiments, aliphatic groups contain 1-6 aliphatic carbon atoms, for example C₁₋₆alkyl, and in yet other embodiments aliphatic groups contain 1-4 aliphatic carbon atoms, for example, C₁₋₄alkyl. In some embodiments, “cycloaliphatic” (or “carbocycle” or “cycloalkyl”) refers to a monocyclic C₃₋₈ hydrocarbon or bicyclic C₈₋₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heterocycle”, “heterocyclyl”, “heterocycloaliphatic”, or “heterocyclic” as used herein means non-aromatic, monocyclic, bicyclic, or tricyclic ring systems in which one or more ring members is an independently selected heteroatom. In some embodiments, the “heterocycle”, “heterocyclyl”, “heterocycloaliphatic”, or “heterocyclic” group has three to fourteen ring members in which one or more ring members is a heteroatom independently selected from oxygen, sulfur, nitrogen, or phosphorus, and each ring in the system contains 3 to 7 ring members.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR+ (as in N-substituted pyrrolidinyl)).

The term “unsaturated”, as used herein, means that a moiety has one or more units of unsaturation.

The term “alkoxy”, or “thioalkyl”, as used herein, refers to an alkyl group, as previously defined, attached to the principal carbon chain through an oxygen (“alkoxy”) or sulfur (“thioalkyl”) atom, for example C₁₋₄alkoxy refers to the alkoxyl group, methoxy, ethyoxy, propoxy, and butoxy, including for propoxy and butoxy, the straight and branched structures, that is i-propoxy and n-propoxy; and n-butoxy, i-butoxy and sec-butoxy.

The terms “haloalkyl”, “haloalkenyl” and “haloalkoxy” means alkyl, alkenyl or alkoxy, as the case may be, substituted with one or more halogen atoms. The term “halogen” or “halo” means F, Cl, Br, or I.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring”. The term “aryl” also refers to heteroaryl ring systems as defined hereinbelow.

The term “heteroaryl”, used alone or as part of a larger moiety as in “heteroaralkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from halogen; —R^{o}; —OR^{o}; —SR^{o}; 1,2-methylene-dioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —NR^{o}C(O)R^{o}; —NR^{o}C(O)N(R^{o})₂; —NR^{o}CO₂R^{o}; —NR^{o}NR^{o}C(O)R^{o}; —NR^{o}NR^{o}C(O)N(R^{o})₂; —NR^{o}NR^{o}CO₂R^{o}; —C(O)C(O)R^{o}; —C(O)CH₂C(O)R^{o}; —CO₂R^{o}; —C(O)R^{o}; —C(O)N(R^{o})₂; —OC(O)N(R^{o})₂; —S(O)₂R^{o}; —SO₂N(R^{o})₂; —S(O)R^{o}; —NR^{o}SO₂N(R^{o})₂; —NR^{o}SO₂R^{o}; —C(═S)N(R^{o})₂; —C(═NH)—N(R^{o})₂; or —(CH₂)₀₋₂NHC(O)R^{o} wherein each independent occurrence of R^{o} is selected from hydrogen, optionally substituted C₁₋₆ aliphatic, an unsubstituted 5-6 membered heteroaryl or heterocyclic ring, phenyl, —O(Ph), or —CH₂(Ph), or, notwithstanding the definition above, two independent occurrences of R^{o}, on the same substituent or different substituents, taken together with the atom(s) to which each R^{o} group is bound, form a 3 to 8 membered cycloalkyl, heterocyclyl, aryl, or heteroaryl ring having 0-3 heteroatoms independently selected from nitrogen, oxygen, or sulfur. Optional substituents on the aliphatic group of R^{o} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄aliphatic), or haloC₁₋₄aliphatic, wherein each of the foregoing C₁₋₄aliphatic groups of R^{o} is unsubstituted.

An aliphatic or a non-aromatic heterocyclic ring may contain one or more substituents. Suitable substituents on the saturated carbon of an aliphatic or heteroaliphatic group, or of a non-aromatic heterocyclic ring are selected from those listed above for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

Optional substituents on the nitrogen of a non-aromatic heterocyclic ring are selected from —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₁₋₂(Ph); optionally substituted —CH═CH(Ph); or an unsubstituted 5-6 membered heteroaryl or heterocyclic ring having one to four heteroatoms independently selected from oxygen, nitrogen, or sulfur, or, notwithstanding the definition above, two independent occurrences of R⁺, on the same substituent or different substituents, taken together with the atom(s) to which each R⁺ group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R⁺ is unsubstituted.

The term “alkylidene chain” refers to a straight or branched carbon chain that may be fully saturated or have one or more units of unsaturation and has two points of attachment to the rest of the molecule.

As detailed above, in some embodiments, two independent occurrences of R^{o} (or R⁺, or any other variable similarly defined herein), are taken together together with the atom(s) to which each variable is bound to form a 3-8-membered cycloalkyl, heterocyclyl, aryl, or heteroaryl ring having 0-3 heteroatoms independently selected from nitrogen, oxygen, or sulfur. Exemplary rings that are formed when two independent occurrences of R⁰ (or R⁺, or any other variable similarly defined herein) are taken together with the atom(s) to which each variable is bound include, but are not limited to the following: a) two independent occurrences of R^{o} (or R⁺, or any other variable similarly defined herein) that are bound to the same atom and are taken together with that atom to form a ring, for example, N(R^{o})₂, where both occurrences of R^{o} are taken together with the nitrogen atom to form a piperidin-1-yl, piperazin-1-yl, or morpholin-4-yl group; and b) two independent occurrences of R^{o} (or R⁺, or any other variable similarly defined herein) that are bound to different atoms and are taken together with both of those atoms to form a ring, for example where a phenyl group is substituted with two occurrences of OR^{o} 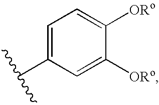
these two occurrences of R^{o} are taken together with the oxygen atoms to which they are bound to form a fused 6-membered oxygen containing ring:

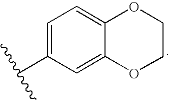

It will be appreciated that a variety of other rings can be formed when two independent occurrences of R^{o} (or R⁺, or any other variable similarly defined herein) are taken together with the atom(s) to which each variable is bound and that the examples detailed above are not intended to be limiting.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

A bond depicted as “----” is a single bond or a double bond.

3. Description of Exemplary Compounds

As described generally above, for compounds of Formula I, Z is C═O, and U and V are independently selected from the group consisting of O, S, C═O, —CH₂—, and —NR^{V}—, wherein R² is hydrogen. Accordingly in certain embodiments, where V and U are —NH—, the resulting compounds have the structure of Formula I-A:

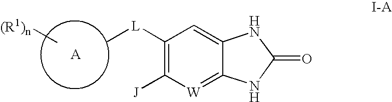

For example, the following compounds that are taught in Table 1 can be described accordingly;

Compound I-6 is a compound of Formula I-A where W is N, J is hydrogen, L is C(O)—NH—(CH²)_{q}—, where q is 0, Ring A is furanyl, and R¹₁ and R¹₂ are C₁₋₆alkyl, that is, methyl and tert-butyl;

Compound I-6 is a compound of Formula I-A where W is N, J is hydrogen, L is C(O)—NH—(CH²)_{q}—, where q is 0, Ring A is furanyl, and R¹₁ and R¹₂ are C₁₋₆alkyl, that is, methyl and tert-butyl;

Compound I-34 is a compound of Formula I-A where W is C, J is hydrogen, L is C(O)—NH—(CH²)_{q}—, where q is 0, Ring A is isoxazolyl, and R¹ is phenyl with R^{1A}, where R^{1A} is chloro; and

Compound I-71 is a compound of Formula I-A where W is C, J is hydrogen, L is C(O)—NH—(CH²)_{q}—, where q is 0, Ring A is pyridinyl, and R¹ is phenyl with R^{1A}, where R^{1A} is C₁₋₆alkyl, that is ethoxy.

In other embodiments, compounds of Formula I, where V is O and U is —NH—, and the resulting compounds have the structure of Formula I-B: 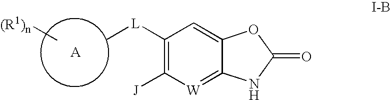
For example, the following compounds that are taught in Table 1 can be described accordingly:

Compound I-25 is a compound of Formula I-B where W is C, J is hydrogen, L is C(O)—NH—(CH₂)_{q}—, where q is 0, Ring A is phenyl, and R¹ is C₁₋₆alkyl, that is tert-butyl; and

Compound I-30 is a compound of Formula I-B where W is C, J is hydrogen, L is —NH—C(O)—NH—, Ring A is phenyl, and R¹ is C₁₋₆alkyl, that is tert-butyl.

In other embodiments, compounds of Formula I, where V is —NH— and U is O, the resulting compounds have the structure of Formula I-C: 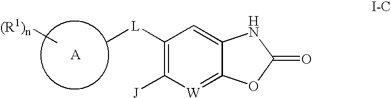
For example, the following compounds that are taught in Table 1 can be described accordingly:

Compound I-11 is a compound of Formula I-C where W is C, J is hydrogen, L is C(O)—NH—(CH₂)_{q}—, where q is 0, Ring A is furanyl, and R¹₁ is C₁₋₆alkyl, that is methyl, and R¹₂ is phenyl, where R^{1A} is tert-butyl;

Compound I-13 is a compound of Formula I-C where W is C, J is hydrogen, L is —NH—C(O)—NH—, Ring A is furanyl, R¹₁ is —CF₃ and R¹₂ is phenyl, where R^{1A} is tert-butyl; and

Compound I-118 is a compound of Formula I-C where W is C, J is hydrogen, L is C(O)—NH—(CH²)_{q}—, where q is 0, Ring A is isoxazolyl, and R¹ is phenyl, where R^{1A} is tert-butyl.

In other embodiments, compounds of Formula I, where V is S and U is —NH—, the resulting compounds have the structure of Formula I-D: 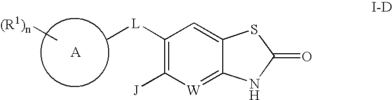
For example the following compound that are taught in Table 1 can be described accordingly:

Compound I-14 is a compound of Formula I-D where W is C, J is hydrogen, L is C(O)—NH—(CH₂)_{q}, where q is 0, Ring A is furanyl, and R¹₁ is C₁₋₆alkyl, that is methyl, and R¹₂ is phenyl, where R^{1A} is tert-butyl; and

Compound I-21 is a compound of Formula I-D where W is C, J is hydrogen, L is C(O)—NH—(CH₂)_{q}—, where q is 0, Ring A is phenyl, and R¹ is tert-butyl.

In other embodiments, compounds of Formula I, where V is —NH— and U is S, the resulting compounds have the structure of Formula I-E:

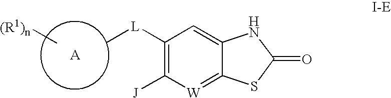

In other embodiments, compounds of Formula I, where V is —CH₂—, Z is N, and U is N, the resulting compounds have the structure of Formula I-F: 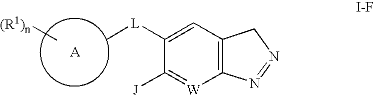
For example, the following compound that is taught in Table 1 can be described accordingly: Compound I-23 is a compound of Formula I-F where W is C, J is hydrogen, L is C(O)—NH—(CH²)_{q}—, where q is 0, Ring A is phenyl, and R¹ is C₁₋₆alkyl, that is tert-butyl.

In other embodiments, compounds of Formula I, where V is N, Z is N, and U is —CH₂—, the resulting compounds have the structure of Formula I-G: 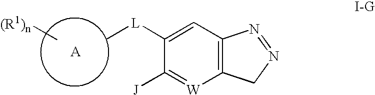
For example, the following compound that is taught in Table 1 can be described accordingly:

Compound I-23 is a compound of Formula I-G where W is C, J is hydrogen, L is C(O)—NH—(CH²)_{q}—, where q is 0, Ring A is phenyl, and R¹ is C₁₋₆alkyl, that is tert-butyl.

According to another embodiment, the present invention provides compounds of formula II useful as VR1 receptor modulators: 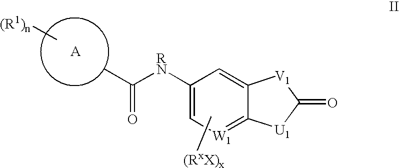
or a pharmaceutically acceptable salt thereof, wherein:

W₁ is CH or N;

V₁ and U₁ each is independently selected from O, S, or NR;

R is hydrogen or an optionally substituted C₁₋₈ aliphatic group;

A₁ is an optionally substituted 3-7 membered monocyclic, heterocyclic or heteroaryl ring;

u is 0-5;

x is 0-3;

U and X each is independently a bond or is an optionally substituted C₁-C₆ alkylidene chain wherein up to two methylene units of V are optionally and independently replaced by —CO—, —CS—, —COCO—, —CONR′—, —CONR′NR′—, —CO₂—, —OCO—, —NR^{o}CO₂—, —O—, —NR^{o}CONR′—, —OCONR′—, —NR′NR′, —NR′NR^{o}CO—, —NR^{o}CO—, —S—, —SO, —SO₂—, —NR′—, —SO₂NR′—, NR′SO₂—, —NR′SO₂NR′—;

R^{U} and R^{X} each is independently R′, CF₃, halogen, NO₂, or CN; and

R′ is hydrogen or an optionally substituted group selected from a C₁-C₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur.

According to another embodiment, the present invention provides compounds of formula (II), provided that:

(i) when V₁ and U₁ each is NH, R is H, then ring A1 together with —(UR^{U})ᵤ is not thiophen-2-yl, 2-bromofuran-5-yl, 3-(2′,6′-dichlorophenyl)-5-methyl-isoxazol-4-yl, 5-bromopyrimidin-3-yl, pyridin-3-yl, furan-2-yl, or

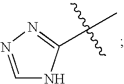

(ii) when V₁ is O, and U₁ is NH, then ring A₁ together with —(UR^{U})ᵤ is not 2-(4′-flurophenoxy)pyridin-3-yl; and

(iii) compound with structure

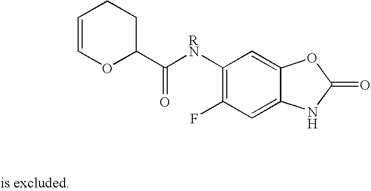

According to one embodiment, V₁ is NR. In one embodiment R is H. Or, V₁ is O. Or, V₁ is S.

According to another embodiment, U₁ is NR. In one embodiment, R is H. Or U₁ is O. Or, U₁ is S.

According to one embodiment, V₁ and U₁ both are NR, preferably, NH. Or, V₁ is NH and U₁ is O. Or, V₁ is NH and U₁ is S. Or, V₁ is O and U₁ is NH. Or, V₁ is S and U₁ is NH.

According to one embodiment, A₁ is selected from any one of the following:

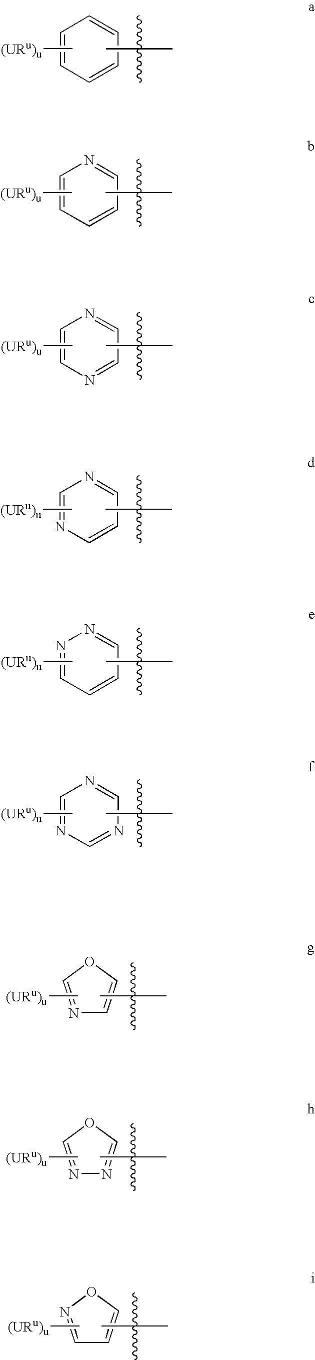

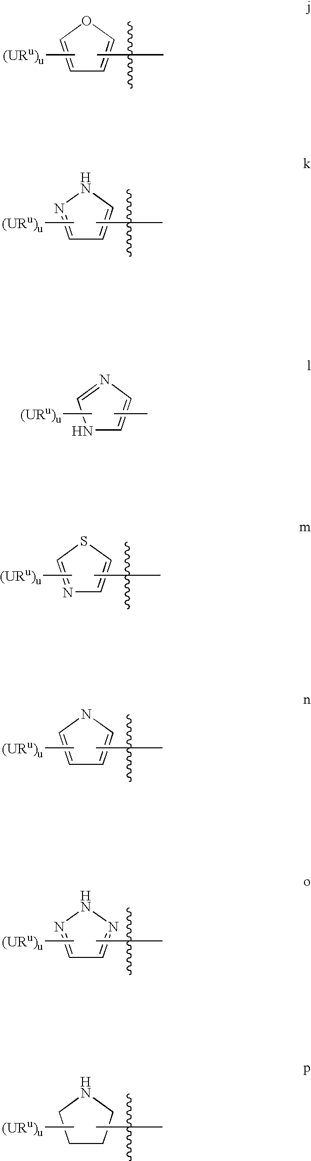

According to one embodiment, A₁ is a or b. Or, A₁ is i, j, k, m, o, or p.

According to one embodiment, R^{X} and R^{U} each is independently R′. Or, each of R^{X} and R^{U} each is independently selected from CF₃, halogen, NO₂, or CN.

According to another embodiment, R′ is hydrogen or an optionally substituted group selected from a C₁-C₈ aliphatic group. In certain embodiments, R′ is H. In certain other embodiments, R′ is optionally substituted C₁-C₆ aliphatic, preferably, optionally substituted C₁-C₄ aliphatic, such as optionally substituted methyl (e.g., benzyl), ethyl, propyl, or butyl.

According to another embodiment, R′ is an optinally substituted 3-8-membered saturated, partially unsaturated, or fully unsaturated monocyclic ring having 0-3 heteroatoms independently selected from nitrogen, oxygen, or sulfur. In certain embodiments, R′ is an optionally substituted 3-6 membered saturated, partially unsaturated, or fully unsaturated monocyclic ring having 0-3 heteroatoms independently selected from nitrogen, oxygen, or sulfur. Examples of such rings include optionally substituted cyclopropyl, cyclopentyl, cyclohexyl, piperidinyl, piperazinyl, morpholinyl, and pyrrolidinyl.

In certain other embodiments, R′ is an optionally substituted 8-12 membered saturated, partially unsaturated, or fully unsaturated bicyclic ring system having 0-5 heteroatoms independently selected from nitrogen, oxygen, or sulfur. Or, two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur.

According to another embodiment, the present invention provides compounds useful as modulators of VR1 receptor: 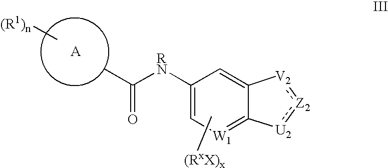
or a pharmaceutically acceptable salt thereof, wherein:
W₂ is CH or N;

one of Z₂, V₂ and U₂ is N; another of Z₂, V₂ and U₂ is NH, and the third of Z₂, V₂ and U₂ is CH;

R is hydrogen or an optionally substituted C₁₋₈ aliphatic group;

A₁ is an optionally substituted 3-7 membered monocyclic, heterocyclic or heteroaryl ring;

u is 0-5;

x is 0-3;

U and X each is independently a bond or is an optionally substituted C₁-C₆ alkylidene chain wherein up to two methylene units of V are optionally and independently replaced by —CO—, —CS—, —COCO—, —CONR′—, —CONR′NR′—, —CO₂—, —OCO—, —NR^{o}CO₂—, —O—, —NR^{o}CONR′—, —OCONR′—, —NR′NR′, —NR′NR^{o}CO—, —NR^{o}CO—, —S—, —SO, —SO₂—, —NR′—, —SO₂NR′—, NR′SO₂—, —NR′SO₂NR′—;

R^{U} and R^{X} each is independently R′, CF₃, halogen, NO₂, or CN; and

R¹ is hydrogen or an optionally substituted group selected from a C₁-C₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur.

According to one embodiment, V₂ is NH, Z₂ is N, and U₂ is CH. According to another embodiment V₂ is N, Z₂ is CH, and U₂ is NH. Or, V₂ is NH, Z₂ is CH, and U₂ is N. Or, V₂ is CH, Z₂ is N, and U₂ is NH.

Representative examples of compounds as described above and herein are set forth below in Table 1.

Table 1. Examples of Compounds of Formula I

TABLE 1
Examples of Compounds of Formula I

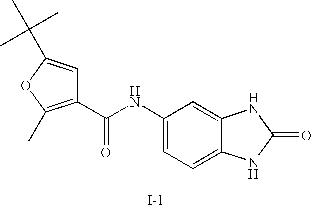

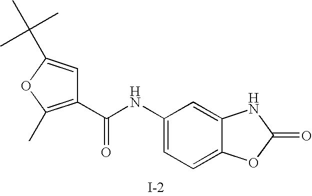

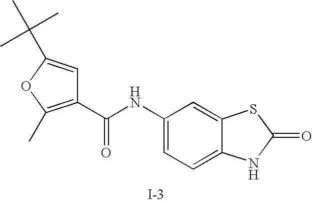

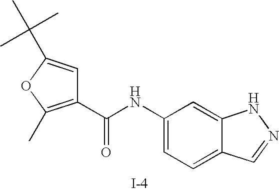

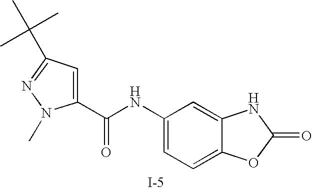

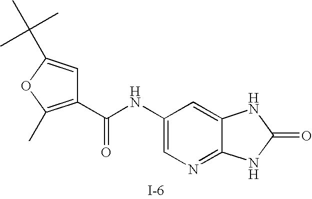

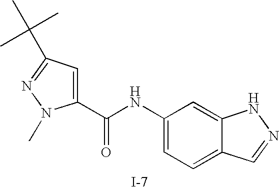

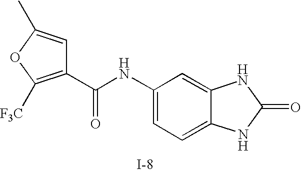

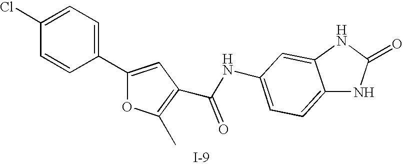

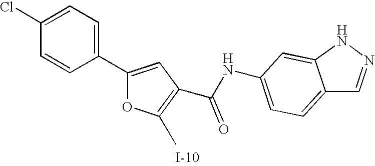

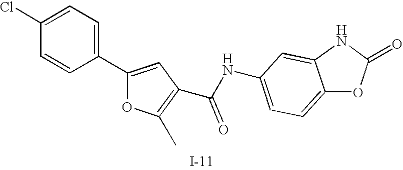

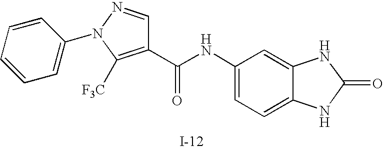

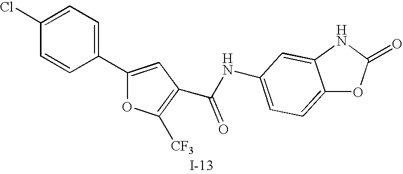

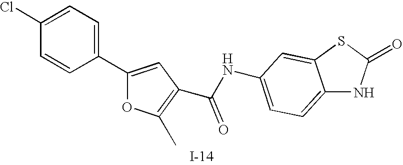

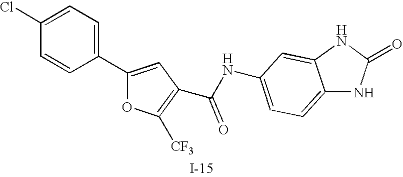

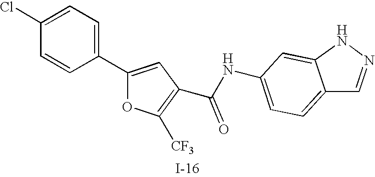

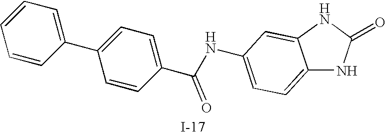

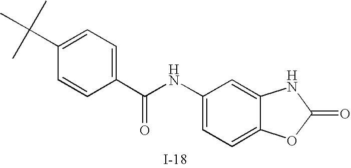

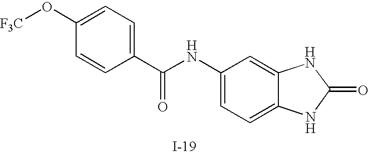

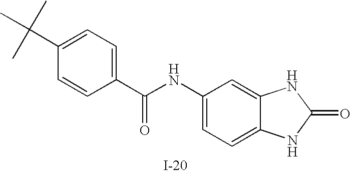

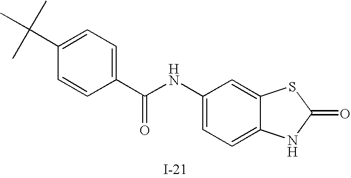

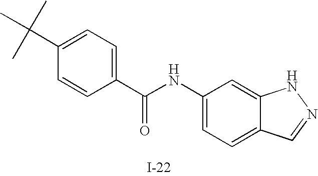

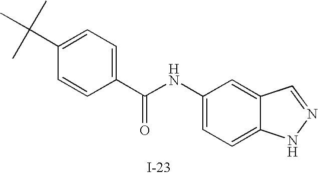

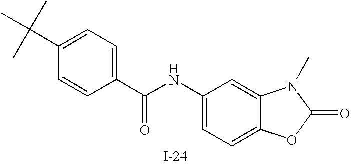

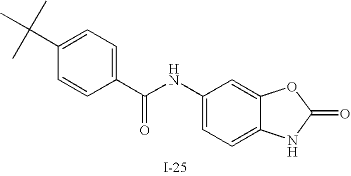

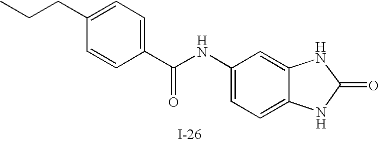

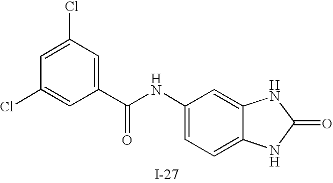

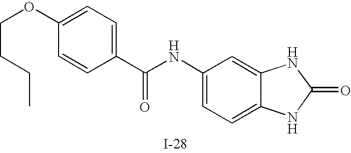

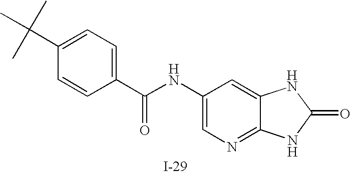

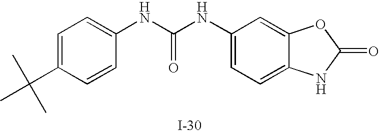

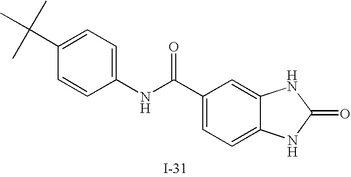

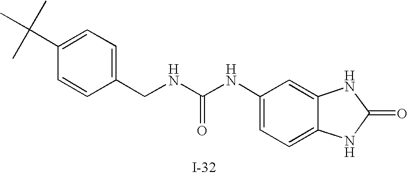

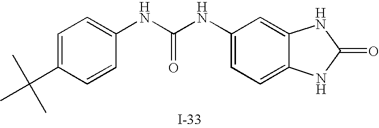

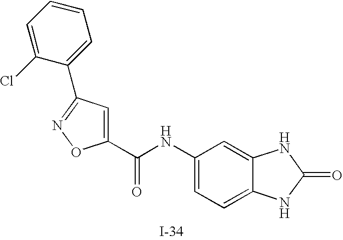

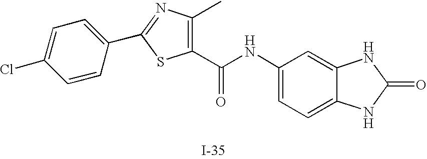

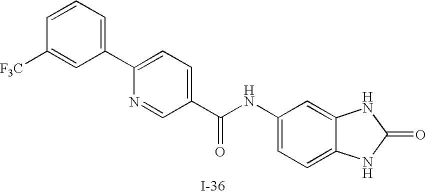

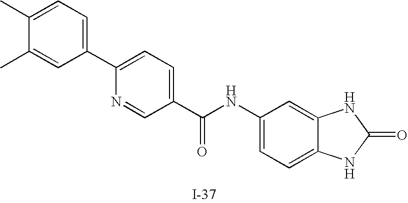

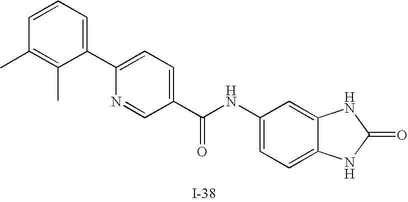

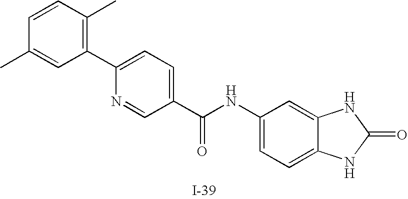

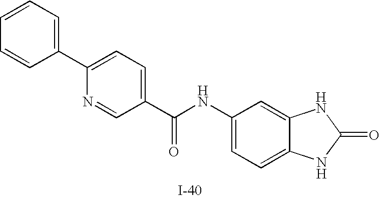

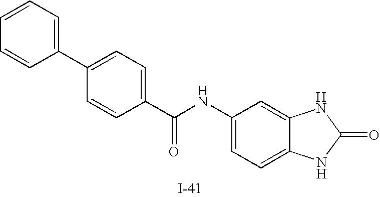

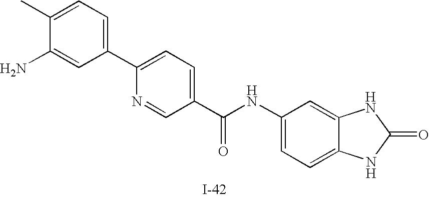

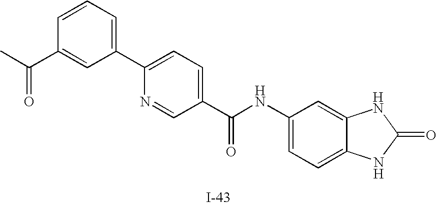

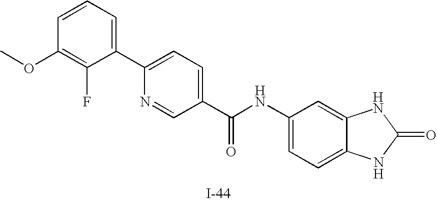

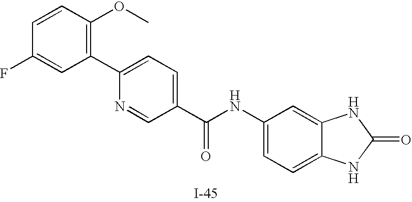

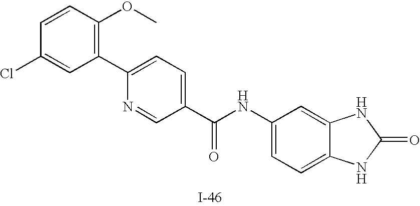

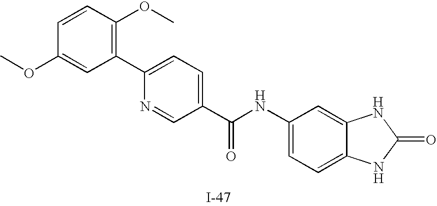

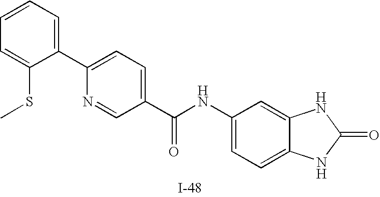

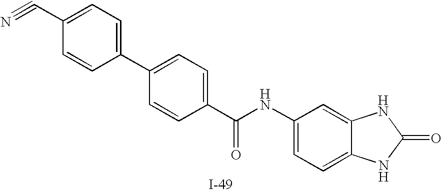

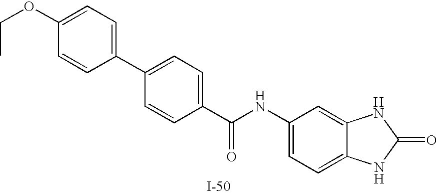

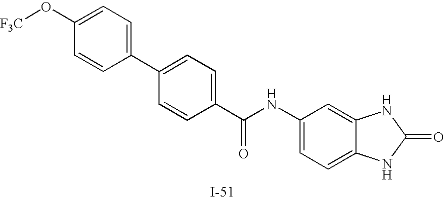

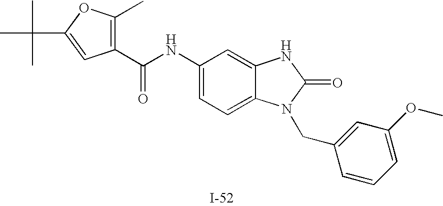

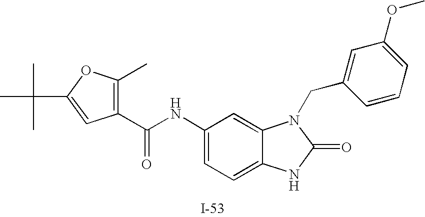

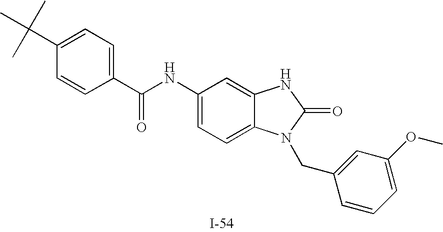

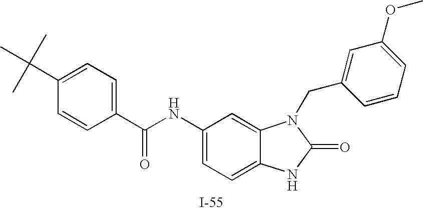

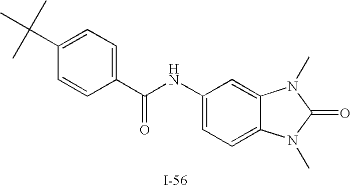

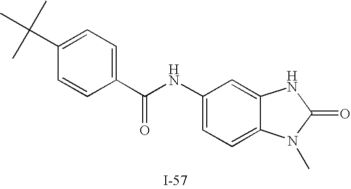

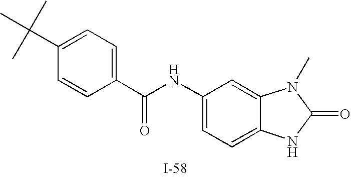

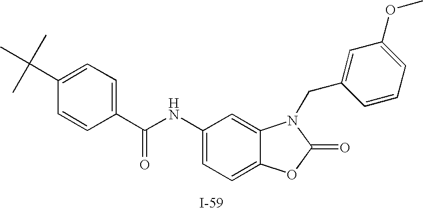

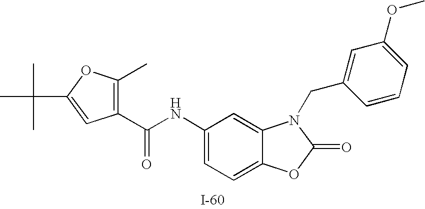

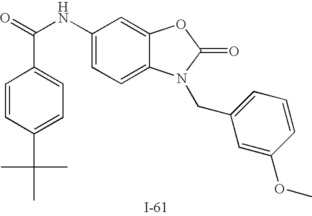

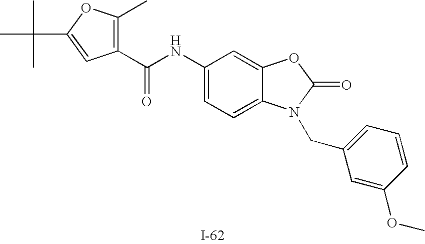

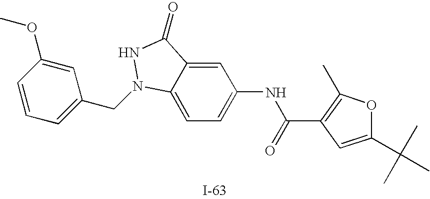

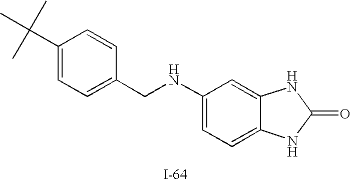

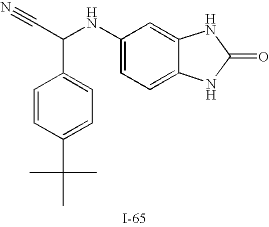

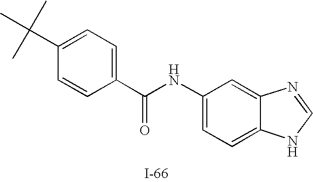

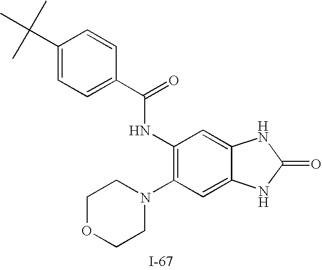

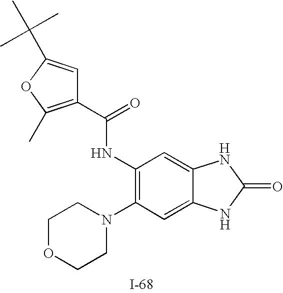

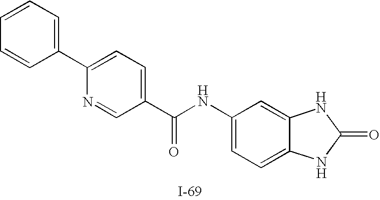

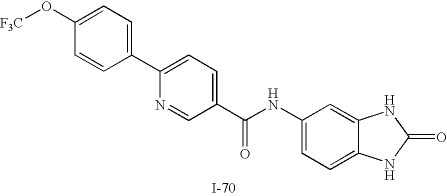

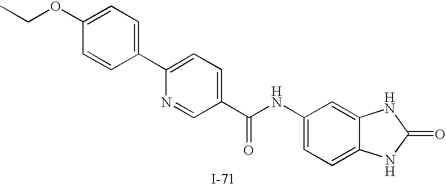

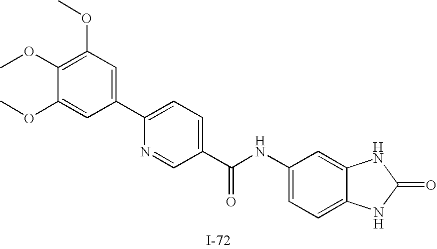

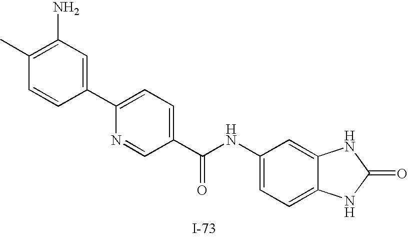

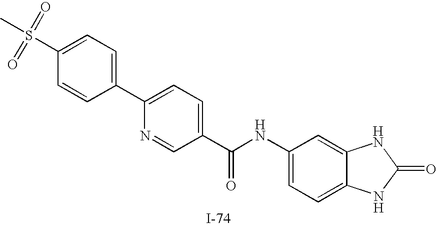

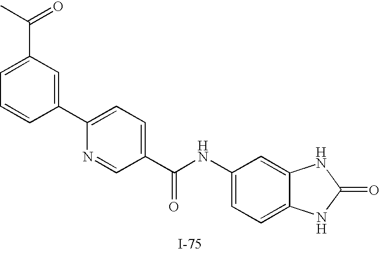

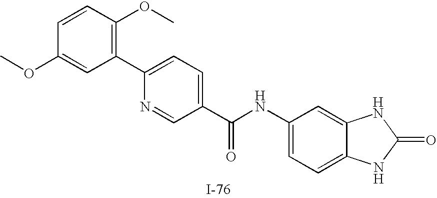

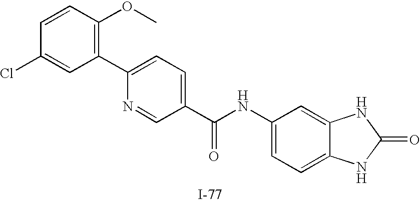

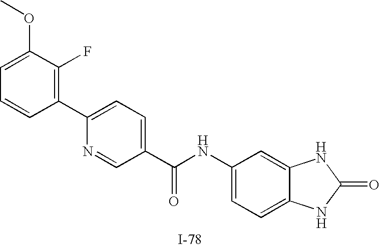

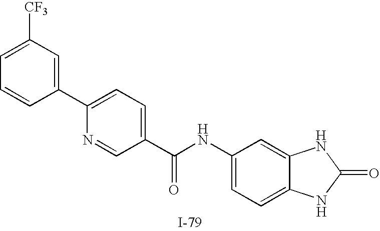

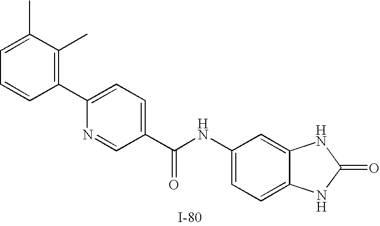

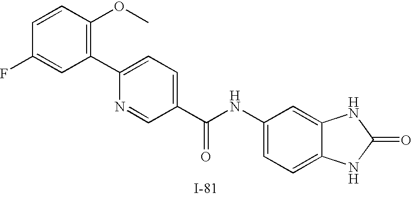

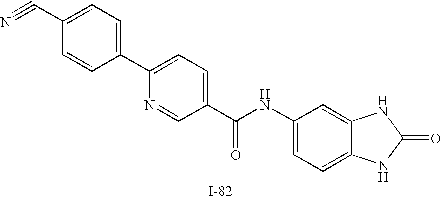

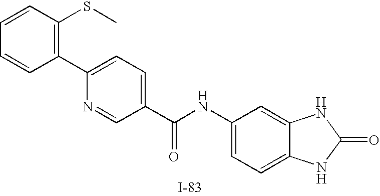

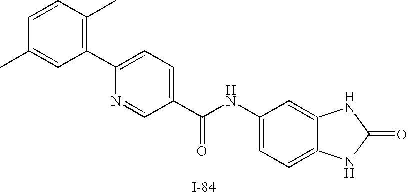

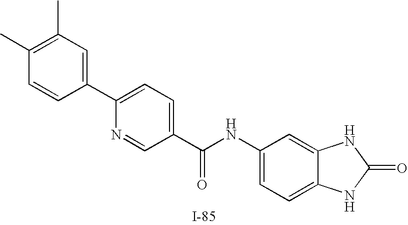

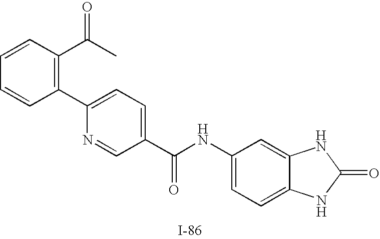

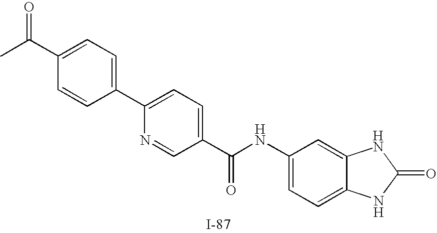

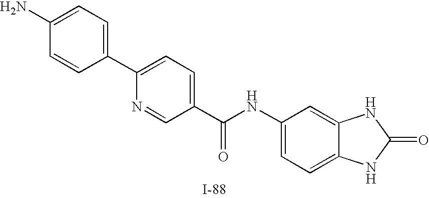

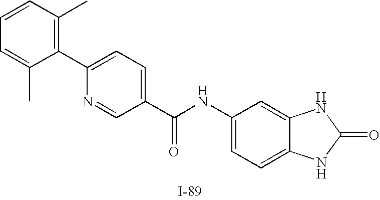

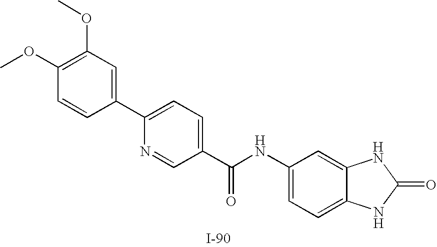

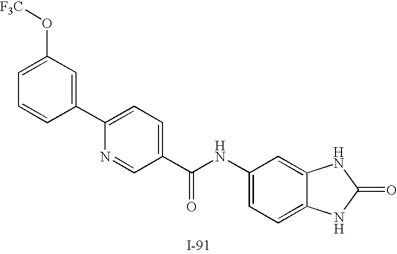

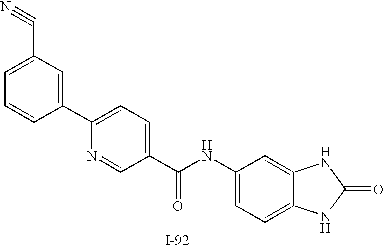

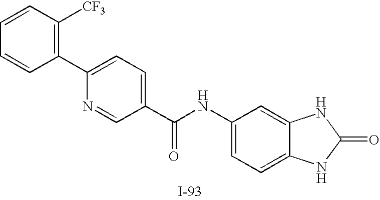

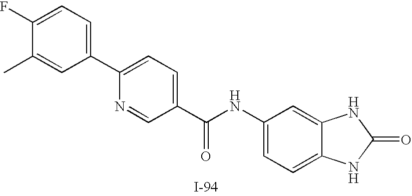

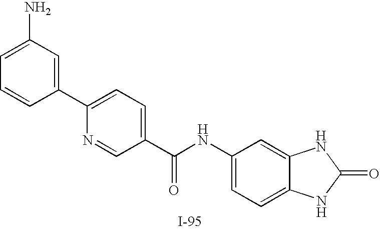

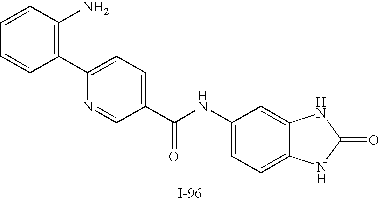

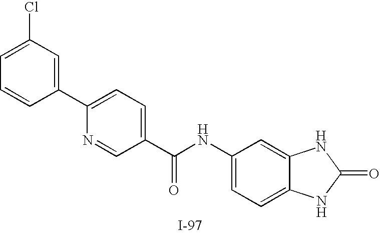

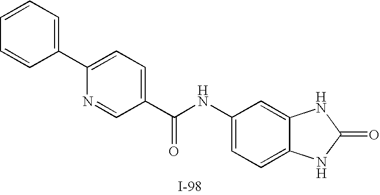

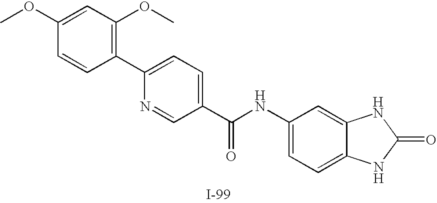

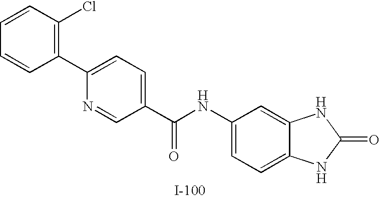

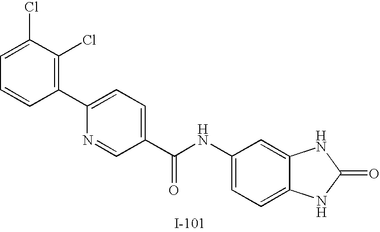

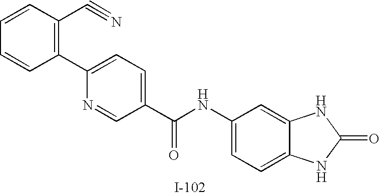

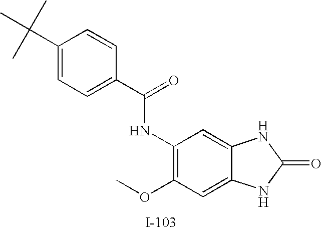

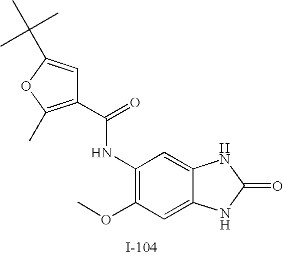

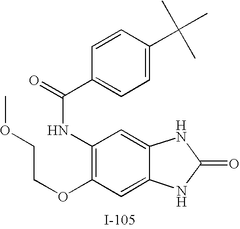

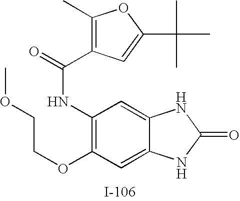

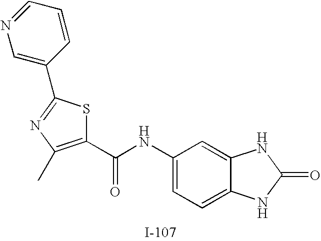

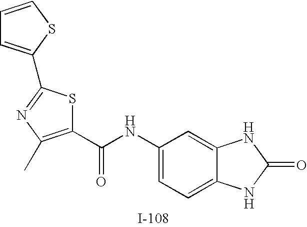

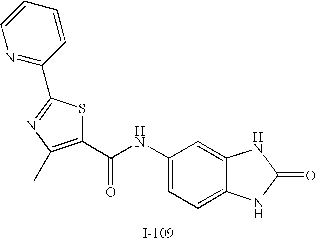

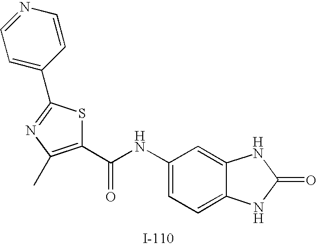

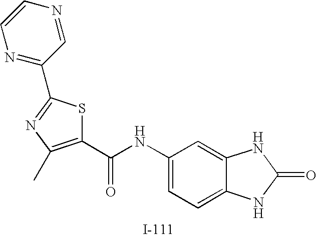

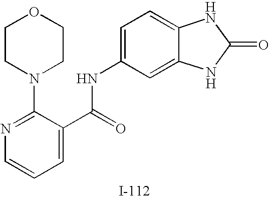

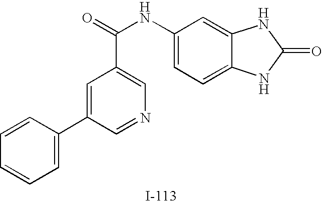

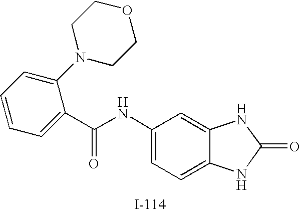

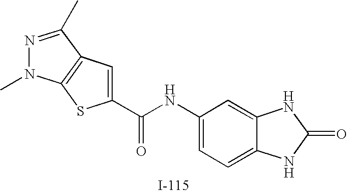

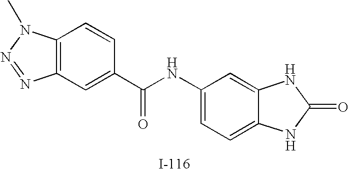

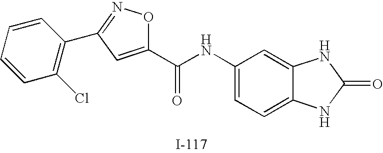

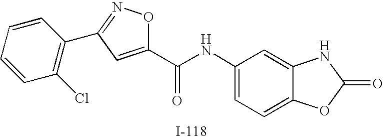

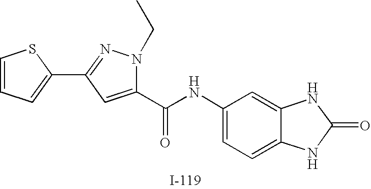

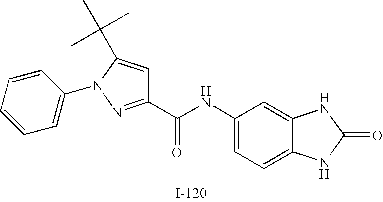

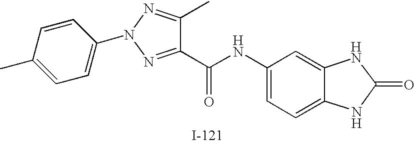

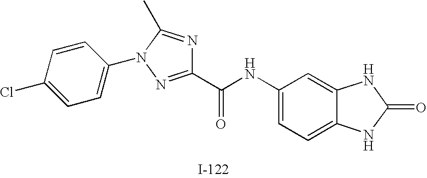

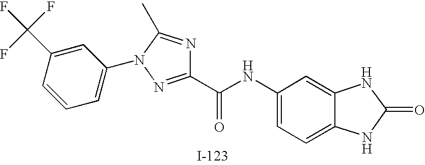

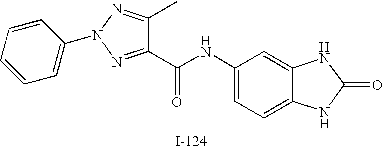

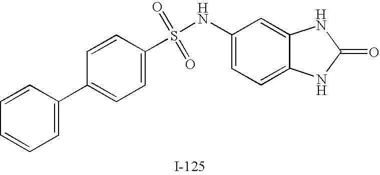

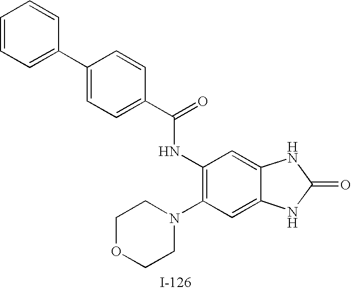

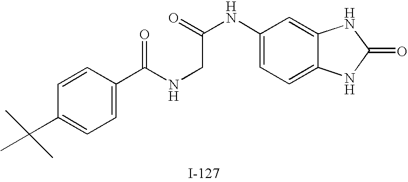

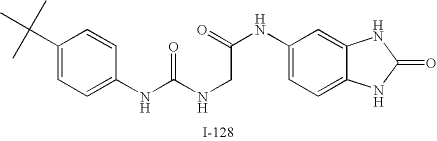

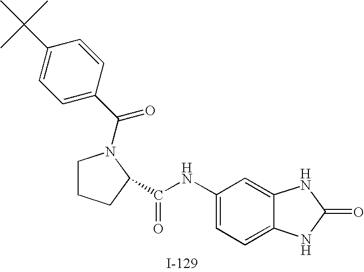

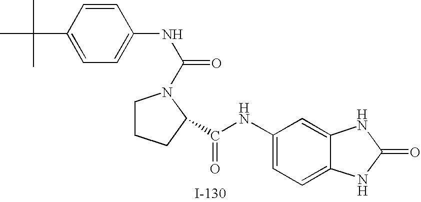

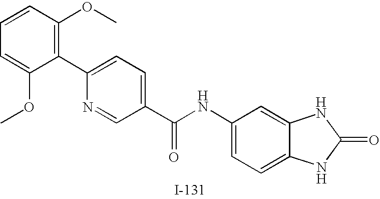

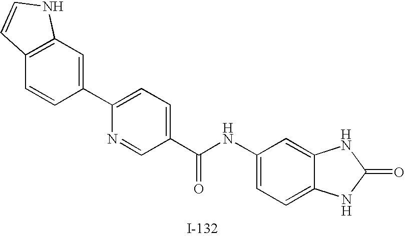

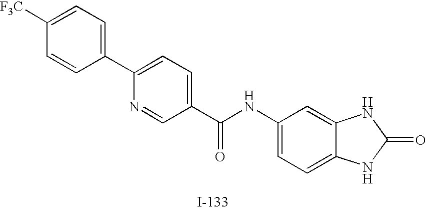

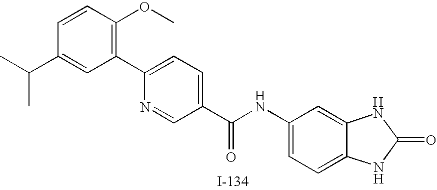

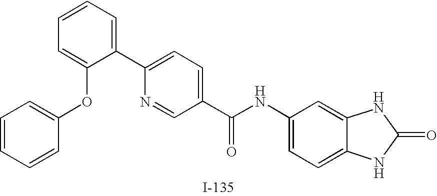

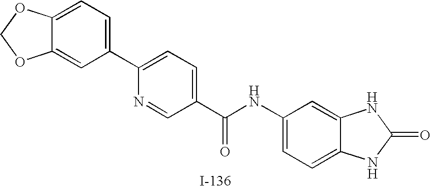

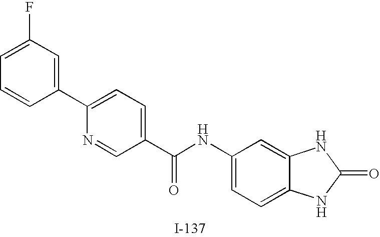

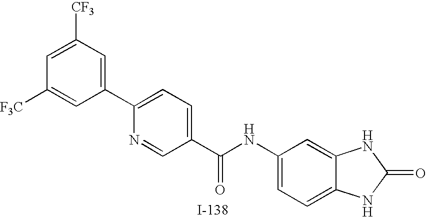

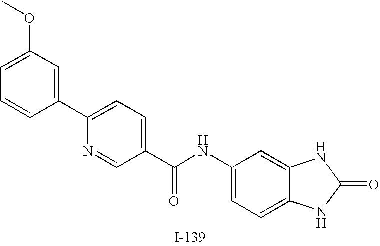

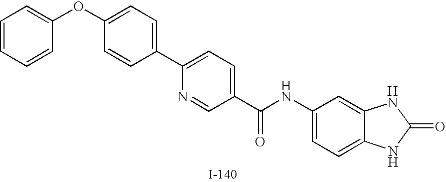

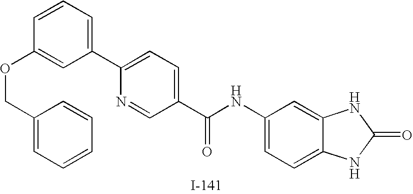

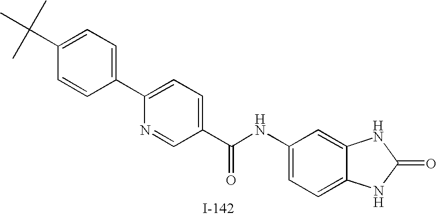

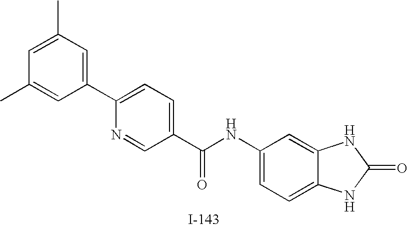

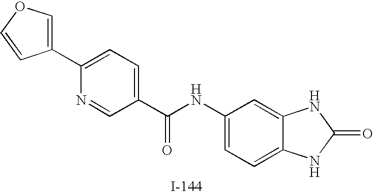

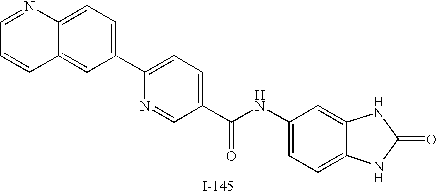

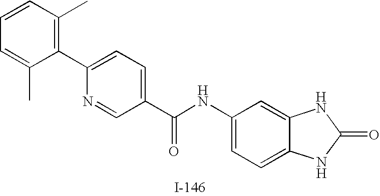

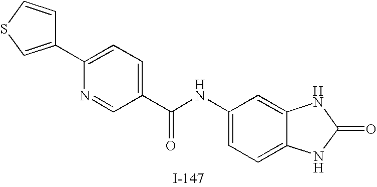

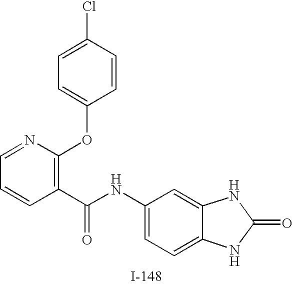

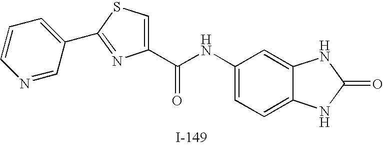

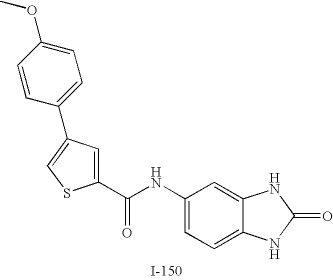

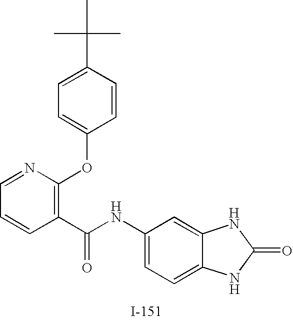

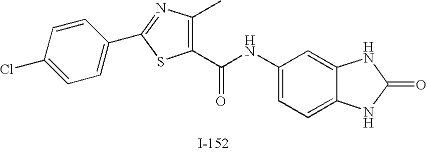

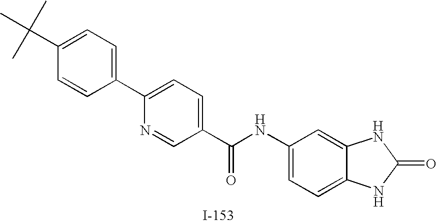

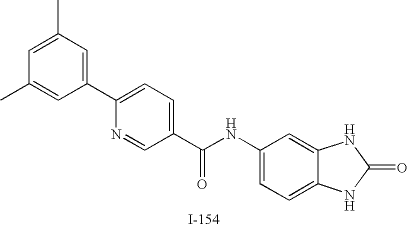

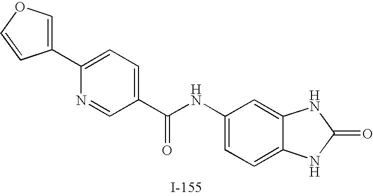

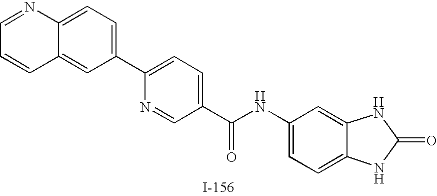

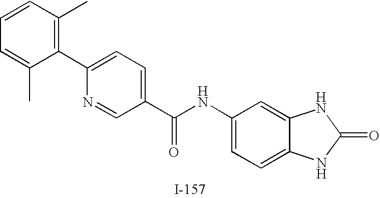

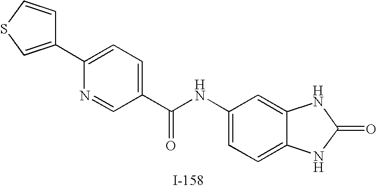

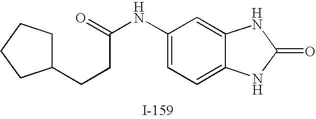

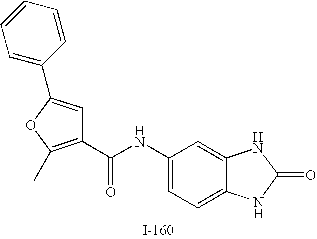

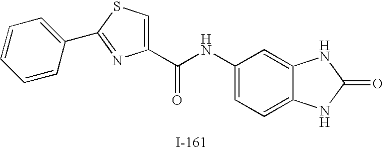

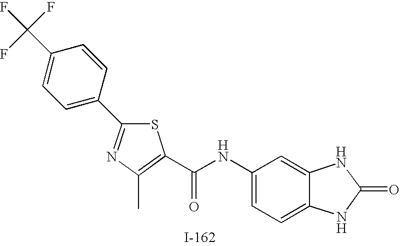

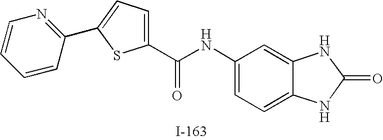

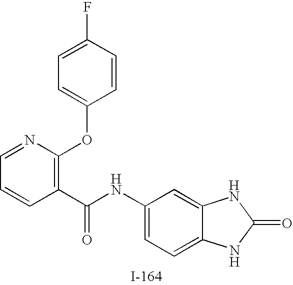

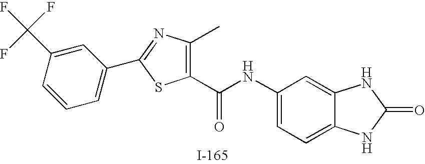

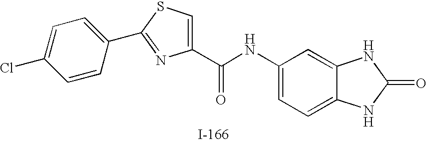

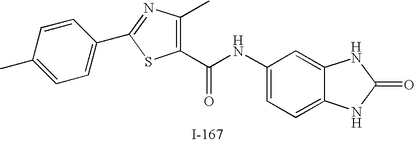

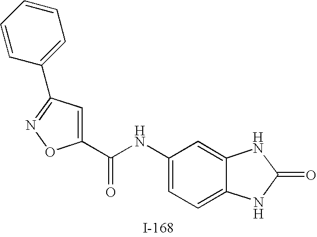

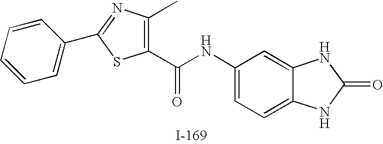

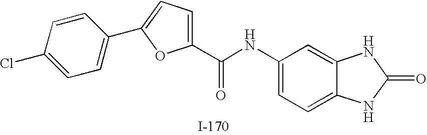

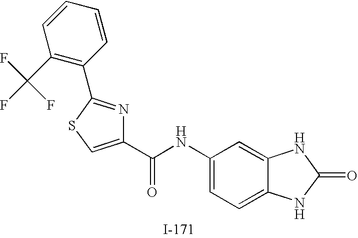

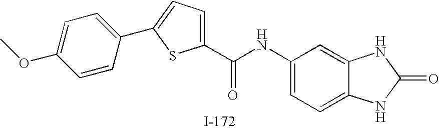

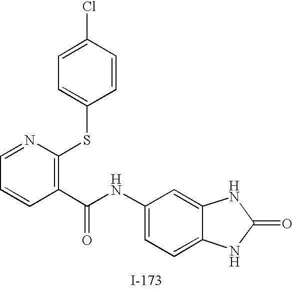

4. General Synthetic Methodology

The compounds of this invention may be prepared in general by methods known to those skilled in the art for analogous compounds, as illustrated by the general scheme below, and the preparative examples that follow.

Scheme 1:

Scheme I below teaches general conditions for the synthesis of compounds of Formula I, in particular the compounds of Formula I-A.

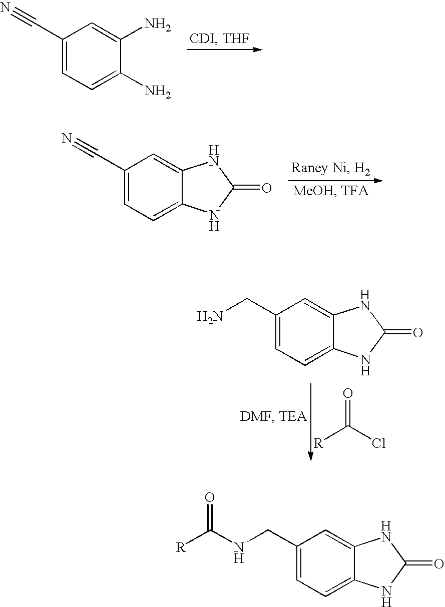

Referring to Scheme 1, the 3,4-diaminobenzonitrile is reacted with CDI in THF. The resulting 2-oxo-2,3-dihydro-1H-benzoimidazole-5-carbonitrile is reduced in the presence of Raney nickel and hydrogen gas to provide the corresponding amine. The amine generated is then reacted in the presence of an appropriate base with one of various kinds of electrophiles, such as, but not limited to carbonyl chlorides, sulfonyl chlorides, isocyanates, isothiocyanates, and the like to provide compounds of Formula I-A. This synthetic methodology is further exemplified in Examples 1 and 2.

Scheme 2

Scheme 2 below teaches the general conditions for the synthesis of compounds of Formula I, in particular the compounds of Formulae I-B and I-C.

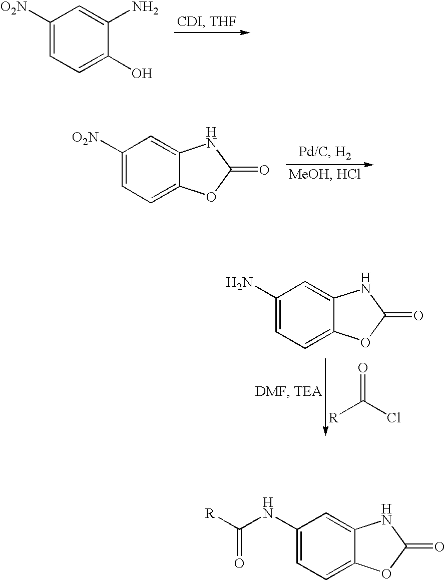

Referring to Scheme 2, the compound 2-amino-4-nitro-phenol is reacted with CDI in THF. The resulting 5-nitro-3H-benzooxazol-2-one is then reduced in the presence of palladium on activated carbon and hydrogen gas to provide the corresponding amine. The amine generated is then reacted in the presence of an appropriate base with one of various kinds of electrophiles, such as, but not limited to carbonyl chlorides, sulfonyl chlorides, isocyanates, isothiocyanates, and the like to provide compounds of Formulae I-B and I-C. This synthetic methodology is further exemplified in Example 3.

Scheme 3

Scheme 3 below teaches the general conditions for the synthesis of compounds of Formula I, in particular those that are varied according to L, Ring A and R^{A}.

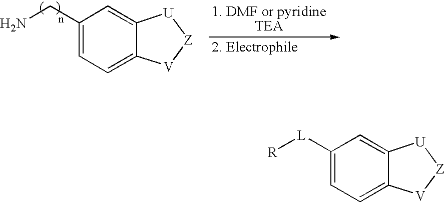

Referring to Scheme 3, an amine may be reacted in the presence of an appropriate base with an electrophile, such as, but not limited to carbonyl chlorides, sulfonyl chlorides, isocyanates, isothiocyanates, and the like to provide compounds of Formula I. This synthetic methodology is further exemplified in Example 4.

Scheme 4

Scheme 4 below teaches the general conditions for the synthesis of compounds of Formula I, in particular those of Formula I-B.

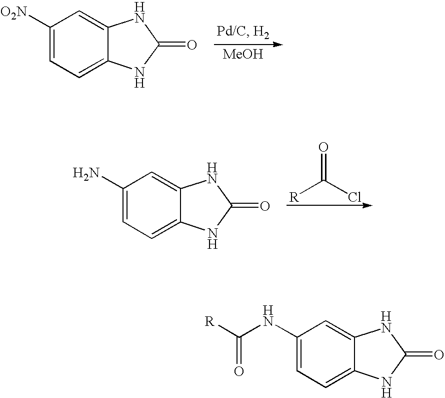

Referring to Scheme 4, the 5-nitro-2-benzimidazolinone is reduced by palladium on activated carbon in the presence of hydrogen gas to provide 5-amino-1,3-dihydro-benzoimidazol-2-one. The amine generated is then reacted in the presence of an appropriate base with one of various kinds of electrophiles, such as but not limited to carbonyl chlorides, sulfonyl chlorides, isocyanates, isothiocyanates, and the like to provide compounds of Formula I-B. This synthetic methodology is further exemplified in Example 5.

Scheme 5

Scheme 5 below teaches the general conditions for the synthesis of compounds of Formula I, in particular those that are varied according to L, Ring A and R^{A}.

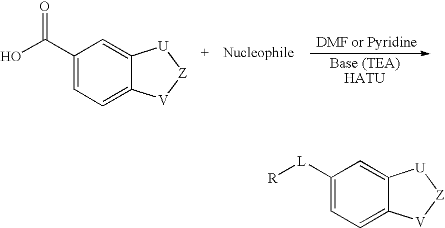

Referring to Scheme 5, a carboxylic acid may be reacted in the presence of an appropriate base, and an activating reagent with a nucleophile, such as but not limited to amines, alcohols, thiols, and the like to provide compounds of Formula I. This synthetic methodology is further exemplified in Example 6.

Scheme 6

Scheme 6 below teaches the general conditions for the synthesis of compounds of Formula I, in particular those that are varied according to R^{A}.

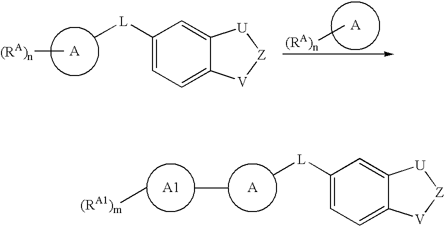

Referring to Scheme 6, compounds of Formula I when Ring A is suitably substituted (X) may be further modified by cross-coupling methodologies to yield bi-aryl, bi-heteroaryl, or aryl-heteroaryl ring systems. This synthetic methodology is further exemplified by Example 7.

Although certain exemplary embodiments are depicted and described above and herein, it will be appreciated that a compounds of the invention can be prepared according to the methods described generally above using appropriate starting materials by methods generally available to one of ordinary skill in the art.

5. Uses, Formulations and Administration Pharmaceutically Acceptable Compositions

As discussed above, this invention relates to VR1 receptor modulators, particularly VR1 receptor functional inhibitors, and to methods for using such modulators for the treatment of diseases including but not limited to pain, inflammatory pain, neuropathic pain, acute pain, chronic pain, post-operative pain, migraine, arthralgia, nerve injury, neurodegeneration, neuropathies, diabetic neuropathy, hyperactive urinary bladder, hypersensitive urinary bladder, urinary incontinence, interstitial cystitis, painful bladder disorders, irritable bowel syndrome, inflammatory bowel disease, inflammatory disease, asthma, chronic obstructive pulmonary disease, digestive tract ulcer, skin irritation, eye irritation, mucous membrane irritation.

Another embodiment of the present invention is a method of

The VR1 receptor gates a nonselective cation channel with high permeability to calcium. Calcium influx can be used to monitor VR1 channel activity. For assessment of VR1, the data presented utilizes a proprietary fluorescence reader (VIPR) in combination with commercially available calcium-sensitive dyes. Upon activation of the VR1 channel by an agonist (e.g. capsaicin), increased fluorescence is detected, and dose-response curves for agonists and antagonists can be generated. The assay utilizes calcium-sensitive dyes in a ratiometric manner. Specifically, excitation/emission ratio of two separate calcium sensing dyes is used to improve the dynamic range of the measurement and to minimize artifacts. The two dyes used are fluo-3AM, a calcium sensitive dye whose emission increases in the presence of calcium, and fura-red, a calcium sensitive dye whose emission decreases in the presence of calcium. The final measurement is the ratio of fluo-3AM and fura-red excitation.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or a active modulator metabolite or residue thereof. As used herein, the term “active modulator metabolite or residue thereof” means that a metabolite or residue thereof is also a modulator to the VR1 receptor.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutically Acceptable Compositions

In yet another method for the treatment or lessening of the severity of diseases including but not limited to pain, inflammatory pain, neuropathic pain, acute pain, chronic pain, post-operative pain, migraine, arthralgia, nerve injury, neurodegeneration, neuropathies, diabetic neuropathy, hyperactive urinary bladder, hypersensitive urinary bladder, urinary incontinence, interstitial cystitis, painful bladder disorders, irritable bowel syndrome, inflammatory bowel disease, inflammatory disease, asthma, chronic obstructive pulmonary disease, digestive tract ulcer, skin irritation, eye irritation, mucous membrane irritation is provided comprising administering an effective amount of a compound, or a pharmaceutically acceptable composition comprising a compound to a subject in need thereof. In certain embodiments of the present invention an “effective amount” of the compound or pharmaceutically acceptable composition is that amount effective for treating or lessening the severity of one or more of the above indicated pain indications.

The compounds and compositions, according to the method of the present invention, may be administered using any amount and any route of administration effective for treating or lessening the severity of one or more of the following conditions, inflammatory pain, neuropathic pain, acute pain, chronic pain, post-operative pain, migraine, arthralgia, nerve injury, neurodegeneration, neuropathies, diabetic neuropathy, hyperactive urinary bladder, hypersensitive urinary bladder, urinary incontinence, interstitial cystitis, painful bladder disorders, irritable bowel syndrome, inflammatory bowel disease, inflammatory disease, asthma, chronic obstructive pulmonary disease, digestive tract ulcer, skin irritation, eye irritation, mucous membrane irritation. The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar--agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating an implantable medical device, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

In order that the invention described herein may be more fully understood, the following examples are set forth. It should be understood that these examples are for illustrative purposes only and are not to be construed as limiting this invention in any manner.

### EXAMPLES

### Example 1

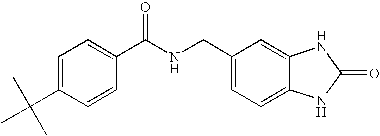

1A. Preparation of 4-tert-Butyl-N-(2-oxo-2,3-dihydro-1H-benzoimidazol-5-ylmethyl)-benzamide

3,4-diaminobenzonitrile (500 mg, 3.76 mmol) was dissolved in 10 mL dry THF and heated to reflux. 1,1′-Carbonyldiimidazole was added portion-wise to the refluxing solution over 25 min. After refluxing for 1 h, the reaction was cooled to ambient temperature and 250 mL of water added. The solvent volume was reduced by rotary evaporation until a precipitate began to form. The reaction mixture was cooled to 0° C. and filtered and dried under vacuum to yield 464 mg (78% yield) of brown solid.

2-Oxo-2,3-dihydro-1H-benzoimidazole-5-carbonitrile (400 mg, 2.51 mmol) was suspended in 20 mL MeOH under nitrogen atmosphere, followed by the addition of 100 mg Raney nickel and 1 mL trifluoroacetic acid. The suspension was exposed to 1 atm of H₂ and heated at 50° C. for 24 h. The Raney nickel was removed by filtration, and the resulting solution concentrated. The residue was dissolved in 20 mL water and washed with 1×50 ML CH₂Cl₂ and 1×50 ethyl acetate. The water layer was evaporated, brought up in minimal MeOH, and precipitated through the addition of cold CH₃CN. The solid was filtered and dried under vacuum to yield 180 mg (28% yield) of 5-aminomethyl-1,3-dihydro-benzoimidazol-2-one as a white powder. ¹H NMR (400 MHz, d₆-DMSO): δ 10.74 (s, 1H), 10.66 (s, 1H), 8.01 (s, 3H), 7.06 (s, 1H), 7.01 (dd, 1H), 6.95 (d, 1H), 4.00 (m, 2H); ESI MS: 146.6 [M—NH₃]+; LC/MS retention time (2-99% CH₃CN/0.05%TFA gradient): 0.80 min.

5-aminomethyl-1,3-dihydro-benzoimidazol-2-one (16.3 mg, 0.1 mmol) was dissolved in a 1:1 mixture of DMF/triethylamine, followed by the drop-wise addition of 19.7 mg (0.1 mmol) 4-tert-butyl-benzoyl chloride. Rapid precipitation of triethylammonium chloride was observed. The reaction was stirred at ambient temperature for 1 hour and then filtered. The crude reaction mixture was purified by reverse phase HPLC (10-99% CH₃CN/0.05%TFA gradient). ¹H NMR (400 MHz, d₆-DMSO): δ 10.52 (s, 1H), 10.51 (s, 1H), 8.91 (t, 1H), 7.82 (d, 2H), 7.47 (d, 2H), 6.84-6.89 (m, 3H), 4.43 (d, 2H), 1.29 (s, 9H); ESI MS: 324.0 [MH]⁺; LC/MS retention time (2-99% CH₃CN/0.05%TFA gradient): 3.40 min.

1B. Additional Prepared Compounds

Using the procedures taught in Example 1A and Scheme 1, the following compounds were prepared:

2-(4-tert-Butyl-phenyl)-N-(2-oxo-2,3-dihydro-1H-benzoimidazol-5-ylmethyl)-acetamide

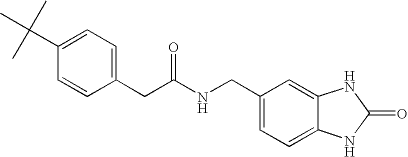

¹H NMR (400 MHz, d₆-DMSO): δ 10.49 (s, 1H), 10.45 (s, 1H), 8.37 (t, 1H), 7.30 (d, 2H), 7.18 (d, 2H), 6.79-6.84 (m, 3H), 4.21 (d, 2H), 3.40 (s, 2H), 1.27 (s, 9H); ESI MS: 338.3 [MH]⁺; LC/MS retention time (10-99% CH₃CN/0.05%TFA gradient): 3.25 min.

4-tert-Butyl-N-(2-oxo-2,3-dihydro-1H-benzoimidazol-5-ylmethyl)-benzenesulfonamide

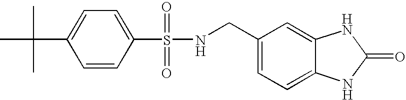

¹H NMR (400 MHz, d₆-DMSO): δ 10.46 (s, 1H), 10.44 (s, 1H), 7.95 (t, 1H), 7.66 (d, 2H), 7.53 (d, 2H), 6.73-6.81 (m, 3H), 3.96 (d, 2H), 1.29 (s, 9H); ESI MS: 385.0 [MH]⁺; LC/MS retention time (10-99% CH₃CN/0.05%TFA gradient): 3.57 min.

5-(4-Chloro-phenyl)-2-methyl-furan-3-carboxylic acid (2-oxo-2,3-dihydro-1H-benzoimidazol-5-ylmethyl)-amide

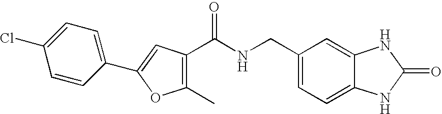

¹H NMR (400 MHz, d₆-DMSO): δ 10.46 (s, 2H), 8.48 (t, 1H), 7.61 (d, 2H), 7.49 (d, 2H), 7.32 (s, 1H), 6.84-6.91 (m, 3H), 4.39 (d, 2H), 2.60 (s, 3H); ESI MS: 382.0 [MH]⁺; LC/MS retention time (10-99% CH₃CN/0.05%TFA gradient): 3.66 min.

### Example 2

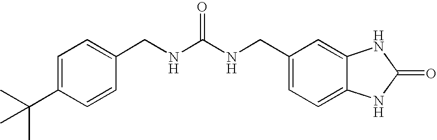

2A Preparation of 1-(4-tert-Butyl-benzyl)-3-(2-oxo-2,3-dihydro-1H-benzoimidazol-5-ylmethyl)-urea

To a solution of 5-aminomethyl-1,3-dihydro-benzoimidazol-2-one trifluoroacetate (28 mg, 0.1 mmol) in 1 mL 1:1 DMF/triethylamine was added 4-tert-butyl benzyl isocyanate (19 mg, 0.1 mmol). After stirring overnight, the crude reaction mixture was purified by reverse phase HPLC (10-99% CH₃CN/0.05%TFA gradient). ¹H NMR (400 MHz, d₆-DMSO): δ 10.48 (s, 1H), 10.43 (s, 1H), 7.32 (d, 2H), 7.16 (d, 2H), 6.80-6.85 (m, 3H), 6.27 (app t, 2H), 4.18 (d, 4H), 1.27 (s, 9H); ESI MS: 353.4 [MH]⁺; LC/MS retention time (10-99% CH₃CN/0.05%TFA gradient): 3.21 min.

### Example 3

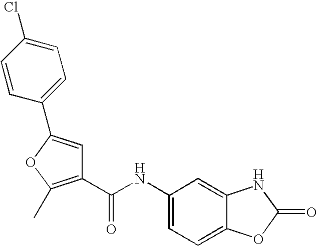

3A. Preparation of 5-(4-Chloro-phenyl)-2-methyl-furan-3-carboxylic acid (2-oxo-2,3-dihydro-benzooxazol-5-yl)-amide

2-Amino-4-nitrophenol (1000 mg, 6.49 mmol) was dissolved in 20 mL dry THF and heated to reflux. 1,1′-Carbonyldiimidazole was added portion-wise to the refluxing solution over 25 min. After refluxing for 1 h, the reaction was cooled to ambient temperature and 50 mL of water added. The solvent volume was reduced by rotary evaporation until a precipitate began to form. The reaction mixture was cooled to 0° C. and filtered and dried under vacuum to yield 990 mg (85% yield) of 5-nitro-3H-benzooxazol-2-one as a brown solid.

5-Nitro-3H-benzooxazol-2-one (500 mg, 2.78 mmol) was suspended in 20 mL MeOH under nitrogen atmosphere, followed by the addition of 100 mg Pd(OH)₂. The suspension was exposed to 1 atm of H₂ and heated at 50° C. for 3 h. The reaction mixture was filtered through Celite, and evaporated to yield an off-white solid. The solid was suspended in CH₃CN and filtered to yield 380 mg (91% yield) of 5-amino-3H-benzooxazol-2-one as an off-white powder.

5-Amino-3H-benzooxazol-2-one one (15.0 mg, 0.1 mmol) was dissolved in a 1:1 mixture of DMF/triethylamine, followed by the portion-wise addition of 25.5 mg (0.1 mmol) 5-(4-chloro-phenyl)-2-methyl-furan-3-carbonyl chloride. Rapid precipitation of triethylammonium chloride was observed. The reaction was stirred at ambient temperature for 1 hour. The crude reaction mixture was purified by reverse phase HPLC (10-99% CH₃CN/0.05%TFA gradient. 1H NMR (400 MHz, d₆-DMSO): δ 11.56 (s, 1H), 9.81 (s, 1H), 7.67-7.69 (m, 3H), 7.53 (d, 2H), 7.47 (s, 1H), 7.33 (dd, 1H), 7.24 (d, 1H), 2.63 (s, 3H); ESI MS: 369.2 [MH]⁺; LC/MS retention time (2-99% CH₃CN/0.05%TFA gradient): 3.88 min.

3B. Additional Prepared Compounds

Using the procedures taught in Example 3A and Scheme 2, the following compound was prepared:

5-tert-Butyl-2-methyl-2H-pyrazole-3-carboxylic acid (2-oxo-2,3-dihydro-benzooxazol-5-yl)-amide

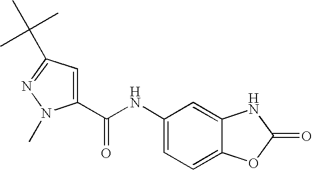

¹H NMR (400 MHz, d₆-DMSO): δ 11.58 (s, 1H) , 10.10 (s, 1H) 8.00 (d, 1H), 7.64 (d, 1H), 7.34 (dd, 1H), 7.24 (d, 1H), 6.91 (s, 1H), 4.02 (s, 3H), 1.28 (s, 9H); ESI MS: 315.4 [MH]⁺; LC/MS retention time (2-99% CH₃CN/0.05%TFA gradient): 3.26 min.

### Example 4

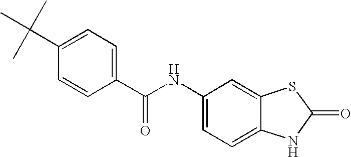

4A. Preparation of 4-tert-Butyl-N-(2-oxo-2,3-dihydro-benzothiazol-6-yl)-benzamide

6-Nitro-3H-benzothiazol-2-one (500 mg, 2.55 mmol) was dissolved in 50 mL MeOH under a nitrogen atmosphere, followed by 500 mg of 10% Pd/C. The mixture was exposed to 1 atm of H₂ for 24 h. The reaction mixture was filtered through Celite, and evaporated to yield 320 mg of 6-amino-3H-benzothiazol-2-one as a light brown solid (76% yield).

6-Amino-3H-benzothiazol-2-one (16.6 mg, 0.1 mmol) was dissolved in a 1:1 mixture of DMF/triethylamine, followed by the drop-wise addition of 4-tert-butyl-benzoyl chloride (19.7 mg, 0.1 mmol). Rapid precipitation of triethylammonium chloride was observed. The reaction was stirred at ambient temperature for 1 hour. The crude reaction mixture was then purified by reverse phase HPLC (10-99% CH₃CN/0.05%TFA gradient). ¹H NMR (400 MHz, d₆-DMSO): δ 11.76 (s, 1H), 10.15 (s, 1H), 8.00 (d, 1H), 7.88 (d, 2H), 7.58 (dd, 1H), 7.54 (d, 2H), 7.10 (d, 1H), 1.33 (s, 9H); ESI MS: 327.2 [MH]⁺; LC/MS retention time (10-99% CH₃CN/0.05%TFA gradient): 3.29 min. ¹H NMR (400 MHz, d₆-DMSO): δ 11.76 (s, 1H), 10.15 (s, 1H), 8.00 (d, 1H), 7.88 (d, 2H), 7.58 (dd, 1H), 7.54 (d, 2H), 7.10 (d, 1H), 1.33 (s, 9H); ESI MS: 327.2 [MH]⁺; LC/MS retention time (10-99% CH₃CN/0.05%TFA gradient): 3.29 min.

4B. Additional Prepared Compounds Using the procedures taught in Example 4A and Scheme 3, the following compounds were prepared.

5-(4-Chloro-phenyl)-2-methyl-furan-3-carboxylic acid (2-oxo-2,3-dihydro-benzothiazol-6-yl)-amide

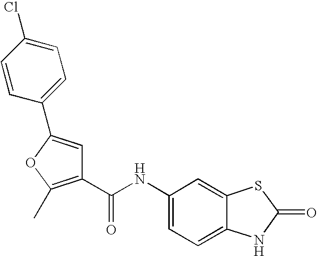

¹H NMR (400 MHz, d₆-DMSO): δ 11.78 (s, 1H), 9.83 (s, 1H), 7.97 (d, 1H), 7.67 (d, 2H), 7.52 (d, 2H), 7.46 (s, 1H), 7.10 (d, 1H), 2.63 (s, 1H); ESI MS: 360.2 [MH]⁺; LC/MS retention time (10-99% CH₃CN/0.05%TFA gradient): 3.46 min.

5-tert-Butyl-2-methyl-furan-3-carboxylic acid (1H-indazol-6-yl)-amide

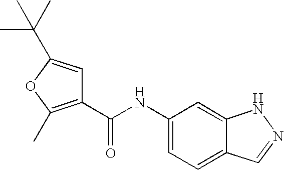

¹H NMR (400 MHz, d₆-DMSO): δ 9.66 (s, 1H), 8.17 (s, 1H), 7.96 (d, 1H), 7.66 (d, 1H), 7.32 (dd, 1H), 6.70 (s, 1H), 2.54 (s, 3H), 1.28 (s, 9H); ESI MS: 298.4 [MH]⁺; LC/MS retention time (2-99% CH₃CN/0.05%TFA gradient): 3.81 min.

5-(4-Chloro-phenyl)-2-methyl-furan-3-carboxylic acid (1H-indazol-6-yl)-amide

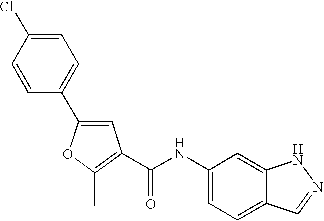

¹H NMR (400 MHz, d₆-DMSO): δ 12.89 (br s, 1H), 9.88 (s, 1H), 8.20 (s, 1H), 7.98 (d, 1H), 7.69 (d, 2H), 7.53 (d, 2H), 7.34 (dd, 1H), 2.65 (s, 3H); ESI MS: 352.4 [MH]⁺; LC/MS retention time (2-99% CH₃CN/0.05%TFA gradient): 3.99 min

4-tert-Butyl-N-(3-oxo-2,3-dihydro-1H-indazol-5-yl)-benzamide

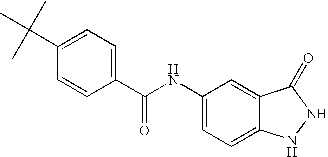

¹H NMR (400 MHz, d6-DMSO): δ 11.17 (br s, 1H) , 10.43 (s, 1H), 10.07 (s, 1H), 8.10 (d, 1H), 7.90 (d, 2H), 7.59 (dd, 1H), 7.54 (d, 2H), 7.25 (d, 1H), 1.33 (s, 9H); ESI MS: 310.3 [MH]⁺; LC/MS retention time (10-99% CH₃CN/0.05%TFA gradient): 3.18 min

4-tert-Butyl-N-(2-oxo-2,3-dihydro-1H-imidazo[4,5-b]pyridin-6-yl)-benzamide

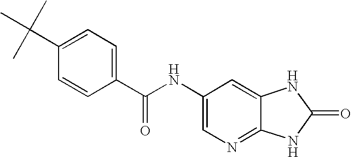

¹H NMR (400 MHz, d₆-DMSO): δ 11.16 (s, 1H), 10.75 (s, 1H), 10.12 (s, 1H), 8.18 (d, 1H), 7.90 (d, 2H), 7.76 (d, 1H), 7.54 (d, 2H), 7.25 (d, 1H), 1.33 (S, 9H); ESI MS: 311.2 [MH]⁺; LC/MS retention time (2-99% CH₃CN/0.05%TFA gradient): 3.75 min

5-tert-Butyl-2-methyl-furan-3-carboxylic acid (2-oxo-2,3-dihydro-1H-imidazo[4,5-b]pyridin-6-yl)-amide

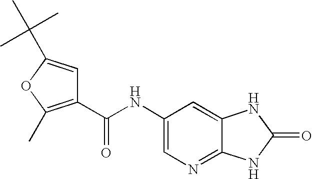

¹H NMR (400 MHz, d₆-DMSO): δ 11.13 (s, 1H), 10.73 (s, 1H), 9.57 (s, 1H), 8.11 (d, 1H), 7.70 (d, 1H), 6.64 (s, 1H), 2.51 (s, 3H), 1.27 (s, 9H); ESI MS: 315.2 [MH]⁺; LC/MS retention time (2-99% CH₃CN/0.05%TFA gradient): 3.79 min

4-tert-Butyl-N-(2,3-dioxo-1,2,3,4-tetrahydro-quinoxalin-6-yl)-benzamide

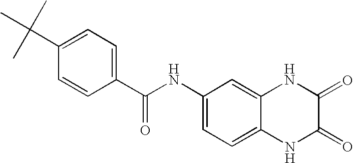

¹H NMR (400 MHz, d₆-DMSO): δ 11.90 (s, 1H) , 11.82 (s, 1H), 10.19 (s, 1H), 7.88 (d, 2H), 7.78 (d, 1H), 7.54 (d, 2H), 7.40 (dd, 1H), 7.09 (d, 1H), 1.33 (s, 9H); ESI MS: 338.2 [MH]⁺; LC/MS retention time (2-99% CH₃CN/0.05%TFA gradient): 3.81 min

### Example 5

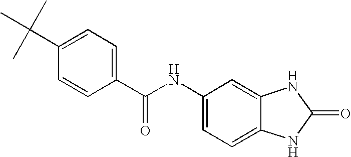

5A. Preparation of 4-tert-Butyl-N-(2-oxo-2,3-dihydro-1H-benzoimidazol-5-yl)-benzamide

5-nitro-2-benzimidazolinone (5 g, 27.9 mmol) was dissolved in 250 mL MeOH under a nitrogen atmosphere, followed by 500 mg of Pd(OH₂). The mixture was exposed to 1 atm of H₂ for 24 h. The reaction mixture was filtered through Celite, and evaporated to yield an off-white solid. The solid was suspended in CH₃CN and filtered to yield 4.0 g (96% yield) of 5-amino-1,3-dihydro-benzoimidazol-2-one as a white solid. ¹H NMR (400 MHz, d₆-DMSO): δ 10.33 (s, 1H), 10.17 (s, 1H), 6.80 (d, 1H), 6.47 (d, 1H), 6.41 (dd, 1H), 4.80 (s, 2H).

5-Amino-1,3-dihydro-benzoimidazol-2-one (14.9 mg, 0.1 mmol) was dissolved in a 1:1 mixture of DMF/triethylamine, followed by the drop-wise addition of 19.7 mg (0.1 mmol) 4-tert-butyl-benzoyl chloride. Rapid precipitation of triethylammonium chloride was observed. The reaction was stirred at ambient temperature for 1 hour and then filtered. The crude reaction mixture was purified by reverse phase HPLC (10-99% CH₃CN/0.05%TFA gradient). ¹H NMR (400 MHz, d₆-DMSO): δ 10.59 (s, 1H), 10.52 (s, 1H), 10.03 (s, 1H), 7.86 (d, 2H), 7.56 (s, 1H), 7.52 (d, 2H), 7.25 (d, 1H), 6.87 (d, 1H), 1.32 (s, 9H); ESI MS: 310.3 [MH]⁺; LC/MS retention time (10-99% CH₃CN/0.05%TFA gradient): 3.14 min.

5B. Additional Prepared Compounds

Using the procedures taught in Example 5A and Scheme 4, the following compounds were prepared:

5-tert-Butyl-2-methyl-furan-3-carboxylic acid (2-oxo-2,3-dihydro-1H-benzoimidazol-5-yl)-amide

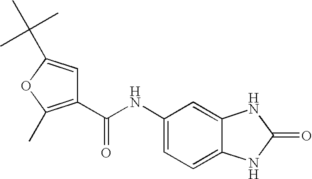

¹H NMR (400 MHz, d₆-DMSO): δ 10.43 (s, 1H), 10.34 (s, 1H), 9.34 (s, 1H), 7.41 (d, 1H), 7.10 (dd, 1H), 6.77 (d, 1H), 6.58 (s, 1H), 2.43 (s, 3H), 1.19 (s, 9H); ESI MS: 314.2 [MH]⁺; LC/MS retention time (2-99% CH₃CN/0.05%TFA gradient): 3.36 min.

4-tert-Butyl-N-(2-oxo-2,3-dihydro-1H-benzoimidazol-5-yl)-benzenesulfonamide

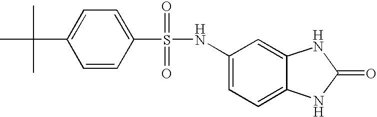

¹H NMR (400 MHz, d₆-DMSO): δ 10.44 (s, 2H), 9.82 (s, 1H), 7.62 (d, 2H), 7.53 (d, 2H), 6.74 (m, 2H), 6.65 (dd, 1H), 1.26 (s, 9H); ESI MS: 346.0 [MH]⁺; LC/MS retention time (2-99% CH₃CN/0.05%TFA gradient): 3.39 min.

### Example 6

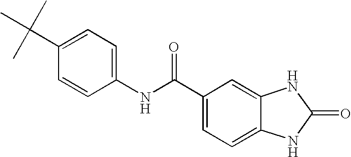

6A. Preparation of 2-Oxo-2,3-dihydro-1H-benzoimidazole-5-carboxylic acid (4-tert-butyl-phenyl)-amide

3,4-Diamino-benzoic acid (1 g, 8.18 mmol) was dissolved in 30 mL dry THF and heated to reflux. 1,1′-Carbonyldiimidazole (1.3 g, 8.18 mmol) was added portion-wise to the refluxing solution over 20 min. After refluxing for 1 h, the reaction was cooled to ambient temperature and 250 mL of water added. The solvent volume was reduced by rotary evaporation until a precipitate began to form. The reaction mixture was cooled to 0° C. and filtered and dried under vacuum to yield 1.05 g (72% yield) of 2-oxo-2,3-dihydro-1H-benzoimidazole-5-carboxylic acid as a brown solid. ¹H NMR (400 MHz, d₆-DMSO): δ 10.92 (s, 1H), 10.77 (s, 1H), 7.61 (d, 1H), 7.47 (s, 1H), 6.99 (d, 1H).

2-Oxo-2,3-dihydro-1H-benzoimidazole-5-carboxylic acid (36 mg , 0.2 mmol), 4-tert-Butyl-phenylamine (28.6 mg, 0.2 mmol), and o-(7-azabenzotriazol-1-yl)-N,N,N′,N′-tetramethyluronium hexafluorophosphate (76 mg, 0.2 mmol) were dissolved in 600 μL dry pyridine and 600 μL triethylamine. The mixture was heated by microwave irradiation for 10 min at 200° C., and then cooled to ambient temperature. The crude reaction mixture was purified by reverse phase HPLC (10-99% CH₃CN/0.05%TFA gradient). ¹H NMR (400 MHz, d₆-DMSO): δ 10.86 (s, 1H), 10.82 (s, 1H), 9.98 (s, 1H), 7.66 (d, 2H), 7.64 (dd, 1H), 7.55 (d, 1H), 7.34 (d, 2H), 7.01 (d, 1H), 1.28 (s, 9H); ESI MS: 310.3 [MH]⁺; LC/MS retention time (10-99% CH₃CN/0.05%TFA gradient): 3.33 min.

### Example 7

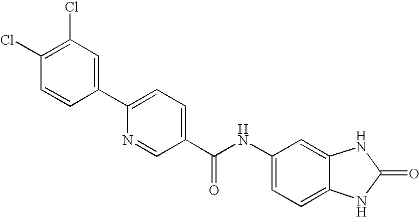

7A. Preparation of 6-(3,4-Dichloro-phenyl)-N-(2-oxo-2,3-dihydro-1H-benzoimidazol-5-yl)-nicotinamide

6-Chloro-N-(2-oxo-2,3-dihydro-1H-benzoimidazol-5-yl)-nicotinamide (28.9 mg, 0.1 mmol), 3,4-dichloroboronic acid (19.1 mg, 0.1 mmol), and tetrakis(triphenylphosphine) palladium(0) (11.6 mg, 0.01 mmol) were suspended in 500 μL acetonitrile and 500 μL 0.4 M Na₂CO₃. The mixture was heated by microwave irradiation for 5 min at 150° C. The crude product was precipitated by the addition of water, filtered, and then purified by reverse phase HPLC (10-99% CH₃CN/0.05%TFA gradient). ¹H NMR (400 MHz, d₆-DMSO): δ 10.65 (s, 1H), 10.58 (s, 1H), 10.36 (s, 1H), 9.18 (d, 1H), 8.44 (d, 1H), 8.41 (dd, 1H), 8.26 (d, 1H), 8.19 (dd, 1H), 7.81 (d, 1H), 7.58 (d, 1H), 7.28 (dd, 1H), 6.90 (d, 1H); ESI MS: 400.2 [MH]⁺; LC/MS retention time (10-99% CH₃CN/0.05%TFA gradient): 3.29 min.

### Example 8

Determination of VR1 Modulation Activity

8A. Measurement of Capsaicin Induced Calcium Influx in the HEK-293 Cell Line Expression Human VR1

Establishment of HEK-293 cell line expressing hVR1

Novel gene expression technology, termed “homologous recombination of endogenous gene enhancement” (or “EDGE”), was used with HEK-293 cells to generate a clonal cell line expressing phenotypic increases in intracellular calcium in response to challenge with capsaicin (a VR1 agonist). U.S. Provisional Patent Application No. 60/408,297 entitled “Methods and Compositions For Rapid Development of Screening Assays” filed on Sep. 5, 2002 teaches this method and is hereby incorporated by reference, and is also attached as Attachment A.

In brief, a plasmid vector containing p-KI Master-SD-Van-YFP was used to introduce the hVR1 gene fragment into the mammalian cell line, HEK-293, as well as the yellow fluorescent protein (YFP) gene fragment. After expansion of these cells using standard techniques, a functional FACS sort was conducted whereby non-YFP expressing cells were selected for. A second round and third round of functional FACS sorting was performed to select single cells which exhibited increased intracellular calcium following exposure to 10 μM capsaicin (calcium was detected using the commercially-available calcium sensitive dye Fluo-3)(method outlined in more detail below). Positive single cell clones were sorted into individual wells of a 96-well plate, and allowed to divide under standard cell culture conditions. In total, 792 clones in nine 96-well plates were tested for capsaicin-evoked increases in intracellular calcium as detected by Fluo-3 on VIPRII. 16 of these were selected based on their responses to 10 μM capsaicin, and one clone (“5B11”) was selected for assay development purposes as this clone exhibited the most robust response to 10 μM capsaicin. Concentration-dependent responses to capsaicin resulted in calculated EC50 values of 106 nM, and in the presence of 50 μM capsazepine, both clones failed to respond to any concentration of capsaicin. These results confirm, functionally and pharmacologically, that the HEK-293 cells are indeed expressing the vanilloid receptor type 1.

Measurement of Ca2+ Influx with VIPR-II

The VR1 receptor gates a nonselective cation channel with high permeability to calcium. Calcium influx can be used to monitor VR1 channel activity. For assessment of VR1, the data presented utilizes a proprietary fluorescence reader (VIPR) in combination with commercially available calcium-sensitive dyes. Upon activation of the VR1 channel by an agonist (e.g. capsaicin), increased fluorescence is detected, and dose-response curves for agonists and antagonists can be generated. The assay utilizes calcium-sensitive dyes in a ratiometric manner. Specifically, excitation/emission ratio of two separate calcium-sensing dyes is used to improve the dynamic range of the measurement and to minimize artifacts. The two dyes used are fluo-3AM, a calcium sensitive dye whose emission increases in the presence of calcium, and fura-red, a calcium sensitive dye whose emission decreases in the presence of calcium. The final measurement is the ratio of fluo-3AM and fura-red excitation.

Measurement of Capsaicin Induced Electrophysiological Responses in DRG Cells from Neonatal Rats

DRG cells from neonatal rat pups were cultured according to published techniques in “Capsaicin sensitivity and voltage-gated sodium currents in colon sensory neurons from rat dorsal root ganglia” Am J Physiol 277(6 Pt 1): G1180-8, and Laird, J. M., V. Souslova, et al. (2002), and standard electrophysiological techniques were used to obtain patch clamp data for compounds of the present invention. Assays measured effects of compounds to activate VR1 responses, and to block responses to application of capsaicin.

Representative compounds of the present invention were assayed according to the protocols taught in this example, and were found to have VR1 modulator activity.

1. A method for modulating VR1, comprising the step of contacting said VR1 with a compound of formula I: 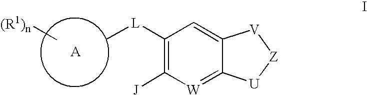
or a pharmaceutically acceptable salt thereof, wherein:
Z is C═O, or N;
V and U are independently selected from the group consisting of O, S, C═O, —CH₂—, —NR²—, wherein R² is —H, C₁₋₄alkyl, or 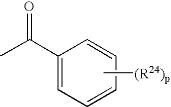
wherein R²A is C₁₋₆alkyl, and p is 0-5;
W is C or N;
J is hydrogen, halo, or C₁₋₄alkoxy;
L is —NH—C (O)—(CH₂)_{q}—, —C(O)—NH—(CH₂)_{q}—, —NH—(CH₂)_{q}—, —(CH₂)_{q}NH— (where q is 0 to 2), —S(O)2NH—, —NH—C(O)—NH—, or —CHR³—C(O)—NH—, wherein R³ is C₁₋₆alkyl;
Ring A is C₃-₇cycloalkyl, phenyl, pyrrolyl, pyrazolyl, imidazolyl, furanyl, thienyl, oxazolyl, isoxazolyl, triazolyl, isothiazolyl, pyridinyl, pyrimidinyl, pyridazinyl, pyrazinyl, piperidinyl, indolyl, indazolyl, benzotriazolyl, benzopyrazolyl, benzimidazolyl, benzthiazolyl, benzisothiazolyl, benzoxazolyl, benzisoxazolyl, benzotriazolyl, thiadiazolyl, benzothienyl, or triazinyl;
R¹ is independently selected from the group consisting of C₁₋₆alkyl, C₁₋₆alkoxy, -halo, —CF₃, —O—CF₃, —NH₂, —NH(C₁-₄alkyl), —N(C₁-₄alkyl)₂, C₁₋₄thioalkyl, —C(O)H, —C(O)OH, —C(O)—R^{1A}, —C(O)OR^{1A}, wherein R^{1A} is C₁₋₆alkyl, and 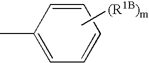
wherein R^{1B} is independently selected from C₁₋₆alkyl, C₁₋₆alkoxy, cyano, and -halo; and m is 0-5; and n is 0-5.

2. The method according to claim 1, wherein L is —C (O)—NH—(CH₂)_{q}—.

3. The according to claim 2, wherein U and V are both NH, and Z is C═O.

4. The method according claim 2, wherein V is S, and U is NH. 12. The compound of claim 10, wherein U is —NH—.

5. The method according to claim 1, wherein Ring A is furanyl or phenyl.

6. A compound having formula II: 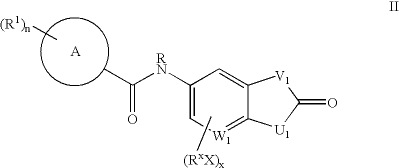
or a pharmaceutically acceptable salt thereof, wherein:
W₁ is CH or N;
V₁ and U₁ each is independently selected from O, S, or NR;
R is hydrogen or an optionally substituted C₁-₈ aliphatic group;
A₁ is an optionally substituted 3-7 membered monocyclic, heterocyclic or heteroaryl ring;
u is 0-5;
x is 0-3;
U and X each is independently a bond or is an optionally substituted C₁-C₆ alkylidene chain wherein up to two methylene units of V are optionally and independently replaced by —CO—, —CS—, —COCO—, —CONR′—, —CONR′NR′—, —CO₂—, —OCO—, —NR′CO₂—, —O—, —NR′CONR′—, —OCONR′—, —NR′NR′, —NR′NR′CO—, —NR′CO—, —S—, —SO, —SO₂—, —NR′—, —SO₂NR′—, NR′SO₂—, —NR′SO₂NR′—;
R^{U} and R^{X} each is independently R′, CF₃, halogen, NO₂, or CN; and
R¹ is hydrogen or an optionally substituted group selected from a C₁-C₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of R¹ are taken together with the atoms to which they are bound to form an optionally substituted 3-12 membered saturated, partially unsaturated, or fully unsaturated monocyclic or bicyclic ring having 0-4 heteroatoms independently selected from nitrogen, oxygen, or sulfur, provided that:
(i) when V₁ and U₁ each is NH, R is H, then ring A₁ together with —(UR^{U})ᵤ is not thiophen-2-yl, 2-bromofuran-5-yl, 3-(2′,6′-dichlorophenyl)-5-methyl-isoxazol-4-yl, 5-bromopyrimidin-3-yl, pyridin-3-yl, furan-2-yl, or 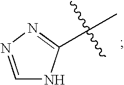
(ii) when V₁ is O, and U₁ is NH, then ring A₁ together with —(UR^{U})ᵤ is not 2-(4′-flurophenoxy)pyridin-3-yl; and
(iii) compound with structure

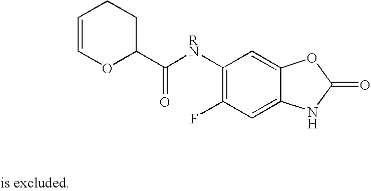

7. The compound according to claim 6, wherein said compound has one or more of the following features:
(i) V₁ is NH, O, or S; and
(ii) U₁ is NH, O, or S.

8. A compound having formula: 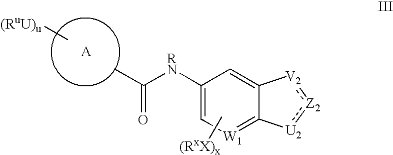
or a pharmaceutically acceptable salt thereof, wherein:
W₂ is CH or N;
one of Z₂, V₂ and U₂ is N; another of Z₂, V₂ and U₂ is NH, and the third of Z₂, V₂ and U₂ is CH;
R is hydrogen or an optionally substituted C₁₋₈ aliphatic group;
A₁ is an optionally substituted 3-7 membered monocyclic, heterocyclic or heteroaryl ring;
u is 0-5;
x is 0-3;
U and X each is independently a bond or is an optionally substituted C₁-C₆ alkylidene chain wherein up to two methylene units of V are optionally and independently replaced by —CO—, —CS—, —COCO—, —CONR′—, —CONR′NR′—, —CO₂—, —OCO—, —NR′CO₂—, —O—, —NR′CONR′—, —OCONR′—, —NR′NR′, —NR′NR′CO—, —NR′CO—, —S—, —SO, —SO₂—, —NR′—, —SO₂NR′—, NR′SO₂—, —NR′SO₂NR′—;
R^{U} and R^{X} each is independently R′, CF₃, halogen, NO₂, or CN; and
R¹ is hydrogen or an optionally substituted group selected from a C₁-C₈ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of R¹ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur.

9. The compound according to claim 8, wherein said compound has one or more of the following features:
(i) V₂ is NH, Z₂ is N, and U₂ is CH;
(ii) V₂ is N, Z₂ is CH, and U₂ is NH;
(iii) V₂ is NH, Z₂ is CH, and U₂ is N; or
(iv) V₂ is CH, Z₂ is N, and U₂ is NH.

10. The compound according to any one of claims 6-9, wherein A₁ is selected from any one of the following:

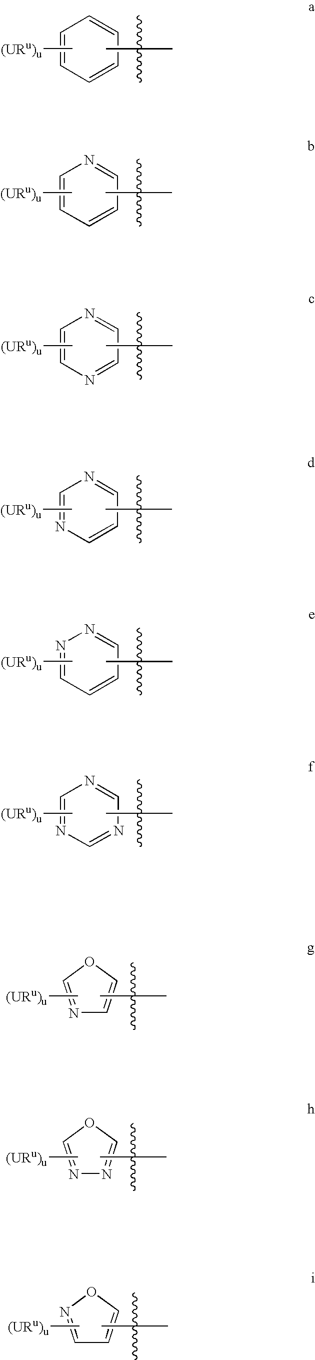

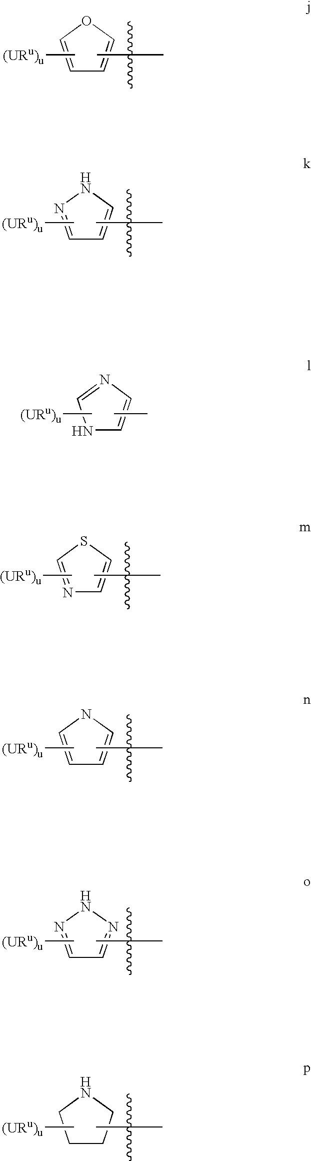

11. The compound according to claim 10, wherein A₁ is a or b.

12. The compound according to claim 11, wherein A₁ is i, j, k, m, o, or p.

13. The compound according to claim 6 or 8, wherein R^{X} and R^{U} each is independently R′.

14. The compound according to claim 13, wherein R¹ is hydrogen or an optionally substituted group selected from a C₁-C₈ aliphatic group.

15. The compound according to claim 13, wherein R′ is an optionally substituted 3-6 membered saturated, partially unsaturated, or fully unsaturated monocyclic ring having 0-3 heteroatoms independently selected from nitrogen, oxygen, or sulfur.

16. The compound according to claim 15, wherein R′ is selected from include optionally substituted cyclopropyl, cyclopentyl, cyclohexyl, piperidinyl, piperazinyl, morpholinyl, and pyrrolidinyl.

17. A pharmaceutical composition comprising a compound according to any one of claims 6-16, and a pharmaceutically acceptable adjuvant or carrier.

18. A method of treating or lessening the severity of a disease in a patient selected from for treating or lessening the severity of one or more of the following conditions, inflammatory pain, neuropathic pain, acute pain, chronic pain, post-operative pain, migraine, arthralgia, nerve injury, neurodegeneration, neuropathies, diabetic neuropathy, hyperactive urinary bladder, hypersensitive urinary bladder, urinary incontinence, interstitial cystitis, painful bladder disorders, irritable bowel syndrome, inflammatory bowel disease, inflammatory disease, asthma, chronic obstructive pulmonary disease, digestive tract ulcer, skin irritation, or eye irritation, mucous membrane irritation, comprising the step of administering to said patient a compound of formula (I), formula (II), or formula (III).
